(19) United States
(12) Patent Application Publication
(10) Pub. No.: US 2025/0199008 A1
(43) Pub. Date: Jun. 19, 2025

(54) COMPOSITION AND METHOD FOR DETECTION OF BIOMOLECULES VIA AROMATIC LABELING USING HETEROCYCLIC COMPOUNDS

(71) Applicants: Life Technologies Corporation, Carlsbad, CA (US); Pierce Biotechnology, Inc., Rockford, IL (US)

(72) Inventors: Thomas Diller, Vista, CA (US); Premchendar Nandhikonda, Rockford, IL (US)

(73) Assignees: Life Technologies Corporation, Carlsbad, CA (US); Pierce Biotechnology, Inc., Rockford, IL (US)

(21) Appl. No.: 18/981,211

(22) Filed: Dec. 13, 2024

Related U.S. Application Data
(60) Provisional application No. 63/610,974, filed on Dec. 15, 2023.

(51) Int. Cl. G01N 33/58; G01N 21/64

(52) U.S. Cl. CPC G01N 33/582; G01N 21/6428; G01N 2021/6439; G01N 2333/936

(57) ABSTRACT

Disclosed herein is a composition comprising a medium and a heterocyclic compound, for detecting a biomolecule in a sample. More specifically, the composition disclosed herein can be used to detect one or more biomolecules comprising one or more aromatic amino acid residues by producing an amino acid conjugate upon associating with the aromatic amino acid residue of the biomolecule. Also disclosed herein are methods of making and using the composition disclosed herein.

### CROSS REFERENCE TO RELATED APPLICATION

This application claims the benefit and priority to the earlier filing date of U.S. Provisional Patent Application No. 63/610,974, filed on Dec. 15, 2023, the entirety of which is incorporated herein by reference.

### FIELD

The present disclosure concerns a composition and kit, and a method of making and using the same for detecting biomolecules in a sample by generating a fluorescent signal upon selective binding of a heterocyclic compound to aromatic amino acid residues of the biomolecule.

### INCORPORATION OF SEQUENCE LISTING

The Sequence Listing is submitted as an XML file in the form of the file named “Sequence.xml” (12,928 bytes), which was created on Dec. 13, 2024, which is incorporated by reference herein.

### BACKGROUND

Proteins are typically detected and characterized using gel electrophoresis, solution quantitation assays, or on solid supports (e.g., filter membranes). Samples with low amounts of protein must be stained before they are localized and identified. For example, stains such as Coomassie Brilliant Blue, silver staining, and zinc are used to determine sample quality, level of separation, and protein load. However, these methods require long multi-step staining procedures, heating, and produce undesirable odors. Thus, staining compositions and methods that enable the visualization of proteins with high sensitivity must have a desirable dynamic range and must not affect protein transfer in downstream applications. For example, staining methods must not react with the gel (e.g., copolymerize with the gel's acrylamide component) and should not interfere with antibody binding through epitope masking.

Gel electrophoresis is widely used for protein separation by placing a sample comprising a protein in an inert support and subjecting it to an electric field to cause the proteins to migrate according to their molecular weight. In one common electrophoresis method, SDS-PAGE, proteins are placed in a polyacrylamide gel and coated with SDS and subjected to an electric field. After the proteins have migrated the bands/spots are stained for visualization. The bands/spots can also be visualized by labeling them with a fluorophore and can be illuminated by a light source that excites the dye-protein complex; however, the gel is first subjected to a solution comprising the dye, washed, rinsed, or subjected to another method that will remove the dye from regions of the support that do not contain the protein. Moreover, the sensitivity of the procedure used also depends upon the protein affinity of the dye and the of the stained protein regions on the support, which typically depends upon how effective the destaining was but nevertheless, excessive destaining results in protein band signal loss. Additionally, the staining and destaining procedures subject the protein to undesirable conditions (e.g., acidic pH levels or organic solvents). Thus, current methods preclude blotting the protein from the gel for downstream analysis or for analyzing separated proteins by mass spectrometry.

Accordingly, there is a need in the art for new compositions and methods that enable fast and sensitive protein visualization, increased efficiency of high-throughput detection without excessive handling, with minimal protein modification, and do not affect protein transfer or downstream antibody binding in western blotting.

### SUMMARY

Disclosed herein is a composition for detecting a biomolecule, comprising: a medium; and a heterocyclic compound comprising at least one ring heteroatom selected from N, O, P, S, B, or Se; or an enantiomer, diastereomer, tautomer, salt, solvate, and/or isotopically substituted derivative thereof; wherein, the heterocyclic compound is selected to associate with an aromatic amino acid residue of the biomolecule and wherein the heterocyclic compound produces a conjugate upon association with the aromatic amino acid residue of the biomolecule, the association taking place between the cyclic heteroatom group and an aromatic ring of the aromatic amino acid residue of the biomolecule. In some aspects of the present disclosure, the heterocyclic compound has a structure according to Formula I,

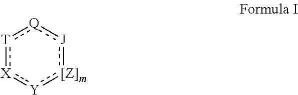

wherein: each of J, Q, T, X, Y, and Z independently is selected from O; P; B; N; S; Se; N(R″) or N⁺[R″], wherein R″ independently is selected from hydrogen, aliphatic, cycloaliphatic, cycloheteroaliphatic, or aryl; or BR^{c} or C(R^{c})ₙ, wherein n is 1 or 2, (i) each R^{c} independently is selected from hydrogen, halo, aliphatic, heteroaliphatic, ether, amino, carbonyl, carboxyl, ketone, aldehyde, isocyanate, cyano, oxime, nitro, nitrile, a salt thereof, or an anionic form thereof, or (ii) for CR^{c}, two R^{c} groups, together with the carbon atoms to which they are attached, form an aliphatic ring system, a heteroaliphatic ring system, or an aromatic ring system; provided that at least one of J, Q, T, X, Y, and Z is O, P, N, S, Se, N(R″), N⁺[R″], or BR^{c}; and m is zero is or one.

Also disclosed herein is a method of making a composition for detecting one or more proteins in a sample, the method comprising adding the composition disclosed herein to a container; adding a monomer and a crosslinker to the container; adding an initiator, promoter, or combination thereof, to the container to promote forming a polymer network derived from the first polymer and second polymer; and adding a buffer to the container.

Also disclosed herein is a method for detecting one or more biomolecules in a sample, comprising adding the sample to a container comprising (i) the composition disclosed herein, (ii) a crosslinked polymer network, and a (iii) buffer; exposing the container to an energy source; and detecting the presence of an amino acid conjugate formed upon association of the heterocyclic compound with the aromatic amino acid residue of the biomolecule.

A kit for detecting one or more biomolecules is also disclosed herein, the kit comprising a container comprising the composition disclosed herein and instructions for using the composition the composition disclosed herein.

The foregoing and other objects, features, and advantages of the present disclosure will become more apparent from the following detailed description, which proceeds with reference to the accompanying figures.

### BRIEF DESCRIPTION OF THE DRAWINGS

FIG. 1A shows the fluorescence emission spectra comparing the reactivity of 4,6-dicholoropyrimidine with a biomolecule with the activity observed for β-butyrolactone and trichloroethanol (TCE).

FIG. 1B is an image showing the sequence for P00698, Gallus gallus (chicken) lysozyme, SEQ ID NO: 1.

FIG. 2A is an illustration showing aspects of folded protein, chicken lysozyme (P00698), a 16.2 kDa protein with 151 amino acids comprising six tryptophan residues (W1-W6).

FIG. 2B is another view of the illustration provided by FIG. 2A.

FIG. 2C is a bar graph showing the peak area intensity of the unmodified fragment of (SEQ ID NO: 3), which compares the abundance in the control (unmodified) sample and the abundance in the unmodified sample.

FIG. 2D is a bar graph showing the peak area intensity of a modified fragment (SEQ ID NO: 4), which compares the abundance in the control (unmodified) sample and the abundance in the 4,6-dichloro-5-fluoropyrimidine (DCFP)-modified sample.

FIG. 2E is a bar graph showing the peak area intensity of an unmodified fragment (SEQ ID NO: 5), which compares the abundance in the control (unmodified) sample and the abundance in the DFCP-sample.

FIG. 2F is a bar graph showing the peak area intensity of a modified fragment (SEQ ID NO: 6), which compares the abundance in the control (unmodified) sample and the abundance in the DCFP-modified sample.

FIG. 2G is a bar graph showing the peak area intensity of a modified fragment (SEQ ID NO: 7), which compares the abundance in the control (unmodified) sample and the abundance in the DCFP-modified sample.

FIG. 2H is a bar graph showing the peak area intensity of a modified fragment (SEQ ID NO: 8), which compares the abundance in the control (unmodified) sample and the abundance in the DCFP-modified sample.

FIG. 2I is a bar graph showing the peak area intensity of a modified fragment (SEQ ID NO: 10), which compares the abundance in the control (unmodified) sample and the abundance in the DCFP-modified sample.

FIG. 3A is a digital image of an electrophoresed Bis-Tris gel (with no UV activation period) comprising 4,6-dichloropyrimidine (0.1%), loaded with four different lysates each in triplicate at 10 μg per lane (HeLa lysate in lanes 1-3; E. coli lysate in lanes 4-6; HEK293 lysate in lanes 7-9; and rat liver lysate in lanes 1-12), and imaged using a manual exposure time of 5 seconds, demonstrating a strong signal intensity for complex samples even in the absence of a UV activation period.

FIG. 3B is a digital image of the same electrophoresed Bis-Tris gel shown in FIG. 3A (but as imaged following a 5-minute UV activation period) comprising 4,6-dichloropyrimidine (0.1%), loaded with four different lysates each in triplicate at 10 μg per lane (HeLa lysate in lanes 1-3; E. coli lysate in lanes 4-6; HEK293 lysate in Lanes 7-9; and rat liver lysate in lanes 10-12), and imaged using an auto (optimal) exposure time of 2.168 seconds, demonstrating a strong signal intensity (arrows indicating a fluorescent signal in the circled area) for complex samples when UV activation is performed and a lower background with comparable signal compared to the shorter exposure time shown in FIG. 3A.

FIG. 3C is a digital image of an electrophoresed Bis-Tris gel (with no UV activation period) comprising trichloro ethanol (TCE) (0.1%), loaded with four different lysates each in triplicate at 10 μg per lane (HeLa lysate in lanes 1-3; E. coli lysate in lanes 4-6; HEK293 lysate in lanes 7-9; and rat liver lysate in lanes 1-12), and imaged using a manual exposure time of 5 seconds, demonstrating a weaker signal intensity for complex samples than that shown in FIG. 3A, which was obtained from a gel comprising 4,6-dichloropyrimidine (0.1%) and imaged without a UV activation period.

FIG. 3D is a digital image of the same electrophoresed Bis-Tris gel shown in FIG. 3C (but imaged following a 5-minute UV activation period) comprising TCE (0.1%), loaded with four different lysates each in triplicate at 10 μg per lane (HeLa lysate in Lanes 1-3; E. coli lysate in lanes 4-6; HEK293 lysate in lanes 7-9, and rat liver lysate in lanes 1-12), and imaged using an auto (optimal) exposure time of 6.275 seconds, demonstrating signal intensity (arrows indicating a fluorescent signal in the circled area) for complex samples comparable to that shown in FIG. 3B with 4,6-dichloropyrimidine (0.1%) yet with an exposure time three times longer.

FIG. 4A is a digital image of an electrophoresed Bis-Tris gel (with no UV activation period) comprising 4,5,6-trichloropyrimidine (0.02%), loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg each protein), and imaged using a manual exposure time of 0.5 seconds, demonstrating signal intensity for both complex samples and purified proteins in the absence of a UV activation period.

FIG. 4B is a digital image of the same electrophoresed Bis-Tris gel shown in FIG. 4A (but imaged following a 45-second UV activation period) comprising 4,5,6-trichloropyrimidine (0.02%), loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg of each protein), and imaged using an auto (optimal) exposure time of 13.319 seconds, demonstrating a strong signal intensity (arrows indicating a fluorescent signal in the circled area) for both complex samples and purified proteins.

FIG. 4C is a digital image of an electrophoresed Bis-Tris gel (with no UV activation period) comprising TCE (0.02%), loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg of each protein), and imaged using a manual exposure time of 0.5 seconds, demonstrating a weak signal intensity for both complex samples and purified proteins in the absence of a UV activation period.

FIG. 4D is a digital image of an electrophoresed Bis-Tris gel (with a 45-second UV activation period) comprising TCE (0.02%), loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg of each protein), and imaged with the gel shown in FIG. 4B using an auto (optimal) exposure time of 13.319 seconds, demonstrating comparatively lower signal intensities for both complex samples and purified proteins relative to the signal intensities observed with 4,5,6-trichloropyrimidine (0.02%) shown in FIG. 4B.

FIG. 4E is a digital image of a PVDF membrane after transferring the electrophoresed Bis-Tris gel of FIG. 4B loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg of each protein) and imaged using an auto (optimal) exposure time of 3.543 seconds, demonstrating that complex and purified proteins derivatized by 4,5,6-trichloropyrimidine transfer from the gel to the membrane and strong signal intensity (arrows indicating a fluorescent signal in the circled area) from derivatized proteins on the membrane.

FIG. 4F is a digital image of a PVDF membrane after transferring the electrophoresed Bis-Tris gel of FIG. 4D loaded with three different lysates (HeLa in lanes 1-3, E. coli in lanes 4-6, and rat liver in lanes 10-12) at three different load amounts (5 μg, 10 μg, and 20 μg) and a five-protein blend (i.e., alcohol dehydrogenase, BSA, carbonic anhydrase, β-lactoglobulin, and lysozyme) at three different load amounts (0.5 μg, 1.0 μg, and 2.0 μg of each protein) and imaged with the membrane shown in FIG. 4E using the same auto (optimal) exposure time of 3.543 seconds, demonstrating comparatively weak signal intensities for both complex samples and purified proteins for the low TCE concentration of 0.02% in the gel, which was not observed in the gel comprising 4,5,6-trichloropyrimidine at 0.02% shown in FIG. 4E.

FIG. 5A is a schematic comparing qualitative results of well-plate labeling with (i) no UV activation period and imaged with a manual exposure time of 2 seconds (top well-plate image); (ii) a 5-minute UV activation period and then imaged with an auto (optimal) exposure time of 0.2 seconds (middle well-plate image); (iii) a 5-minute UV activation period and then imaged using a manual exposure of 3 seconds (bottom well-plate image with dashed lines indicating wells exhibiting fluorescence); wherein the composition comprised (a) unlabeled lysozyme in 1% LDS (wells A1-A3); (b) lysozyme in 1% LDS with 4,6 dichloro-5-fluoropyrimidine (wells B1-B3); (c) lysozyme in 50% MeOH with 2,4,6-trichloropyrimidine (wells C1-C3); (d) unlabeled NAT in 1% SDS (wells D1-D3); (e) NAT in 1% SDS with 4,6 dichloro-5-fluoropyrimidine (wells E1-E3); (f) NAT in 50% MEOH with 2,4,6-trichloropyrimidine (wells F1-F3); (g) lysozyme in 1% LDS with TCE (wells A4-A6); (h) unlabeled lysozyme in 50% MeOH (wells B4-B6); (i) lysozyme in 50% MEOH with 4,6 dichloro-5-fluoropyrimidine (wells C4-C6); (j) NAT in 1% SDS with TCE (wells D4-D6); (k) unlabeled NAT in 50% MeOH (wells E4-E6); (l) NAT in 50% MeOH with 4,6 dichloro-5-fluoropyrimidine (wells F4-F6); (m) lysozyme in 1% LDS with β-butyrolactone (wells A7-A9); (n) lysozyme in 50% MeOH with TCE (wells B7-B9); (o) NAT in 1% SDS with β-butyrolactone (wells D7-D9); (p) NAT in 50% MeOH with TCE (wells E7-E9); (q) lysozyme in 1% LDS with 2,4,6-trichloropyrimidine (wells A10-A12); (r) lysozyme in 50% MeOH (wells B10-B12); (s) NAT in 1% SDS with 2,4,6-trichloropyrimidine (wells D10-D12); and (t) NAT in 50% MeOH (wells E10-E12).

FIG. 5B is a graph showing the emission scans (302 nm excitation) acquired for reaction mixtures following a 5-minute UV activation period and comprised of: (i) 2,4,6-trichloropyrimidine (0.1%) and Lysozyme (0.5 mg/mL) in 1% LDS, (ii) 4,6-dichloro-5-fluoropyrimidine (0.1%) and lysozyme (0.5 mg/mL) in 1% LDS, (iii) 2,4,6-trichloropyrimidine (0.1%) and N-acetyl-tryptophan (0.5 mg/mL) in 1% SDS, (iv) TCE (0.1%) and N-acetyl-tryptophan (0.5 mg/mL) in 50% methanol, (v) TCE (0.1%) and N-acetyl-tryptophan (0.5 mg/mL) in 1% SDS, (vi) 4,6-dichloro-5-fluoropyrimidine (0.1%) and N-acetyl-tryptophan (0.5 mg/mL) in 50% methanol, and (vii) TCE (0.1%) and lysozyme (0.5 mg/mL) in 1% LDS.

FIG. 5C is a bar graph showing the single wavelength emission data (i.e., relative signal intensities of triplicate readings) of reaction mixtures following a 5-minute UV activation period and comprised of (i) 2,4,6-trichloropyrimidine (0.1%) and lysozyme (0.5 mg/mL) in 1% LDS, (ii) 4,6-dichloro-5-fluoropyrimidine (0.1%) and lysozyme (0.5 mg/mL) in 1% LDS, (iii) TCE (0.1%) and NAT (0.5 mg/mL) in 50% methanol, (iv) 2,4,6-trichloropyrimidine (0.1%) and NAT (0.5 mg/mL) in 1% SDS, (v) 4,6-dichloro-5-fluoropyrimidine (0.1%) and NAT (0.5 mg/mL) in 1% LDS, and (vi) TCE (0.1%) and NAT (0.5 mg/mL) in 1% LDS).

FIG. 5D is a digital image of an electrophoresed Bis-Tris gel with no additional UV activation period (samples previously activated for 5 minutes in a 96-well plate prior to loading the samples in the gel), imaged using an auto (optimal) exposure time of 9.526 seconds (arrows indicating a fluorescent signal in the circled area), and comprising: (i) lysozyme (1.0 μg) in 1% LDS, unlabeled control (Lane 1); (ii) lysozyme (1.0 μg) in 1% LDS, TCE-derivatized (lane 2); (iii) lysozyme (1.0 μg) in 1% LDS, β-butyrolactone-derivatized (Lane 3); (iv) lysozyme (1.0 μg) in 1% LDS, 2,4,6-trichloropyrimidine-derivatized (Lane 4); (v) lysozyme (1.0 μg) in 1% LDS, 4,6-dichloro-5-fluoropyrimidine-derivatized (Lane 5); (vi) 10 μL Mark12 Unstained Standard (includes reduced/alkylated lysozyme) (Lane 6); (vii) lysozyme (1.0 μg) in 50% MeOH, unlabeled control (Lane 7); (viii) lysozyme (1.0 μg) in 50% MeOH, TCE-derivatized (Lane 8); (ix) lysozyme (1.0 μg) in 50% MeOH, β-butyrolactone-derivatized (Lane 9); (x) lysozyme (1.0 μg) in 50% MeOH, 2,4,6-trichloropyrimidine-derivatized (Lane 10); (xi) lysozyme (1.0 μg) in 50% MeOH, 4,6-dichloro-5-fluoropyrimidine-derivatized (Lane 11); and (xii) 10 μL Mark12 Unstained Standard (includes reduced/alkylated lysozyme) (Lane 12).

FIG. 5E is a digital image of an electrophoresed Bis-Tris gel as a duplicate to electrophoresed gel of FIG. 5D with no UV activation period (samples previously activated for 5 minutes in a 96-well plate prior to loading the samples in the gel), imaged with the gel shown in FIG. 5D using an auto (optimal) exposure time of 9.526 seconds (arrows indicating a fluorescent signal in the circled area), and comprising: (i) lysozyme (1.0 μg) in 1% LDS, unlabeled control (lane 1); (ii) lysozyme (1.0 μg) in 1% LDS, TCE-derivatized lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 5F is a digital image of the electrophoresed Bis-Tris gel shown in FIG. 5D (imaged following an additional 45-second UV activation period), imaged using an auto (optimal) exposure time of 6.370 seconds (arrows indicating a fluorescent signal in the circled area), and comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 5G is a digital image of the electrophoresed Bis-Tris gel shown in FIG. 5E (imaged following an additional 45-second UV activation period), imaged with the gel shown in FIG. 5F using an auto (optimal) exposure time of 6.370 seconds (arrows indicating a fluorescent signal in the circled area), and comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 5H is a digital image of the Bis-Tris gel shown in FIGS. 5D and 5F after Coomassie (SimplyBlue SafeStain) staining and water de-staining the gel comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% W MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 5I is a digital image of a PVDF membrane (with no further UV activation) after transfer of the Bis-Tris gel shown in FIG. 5E and FIG. 5G and imaged using an auto (optimal) exposure time of 2.764 seconds (arrows indicating a fluorescent signal in the circled area) comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% W MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 6A is a digital image of an electrophoresed Bis-Tris gel (with no UV activation period) comprising 2-chloroethanol (0.05%) loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged using a manual exposure time of 0.5 seconds.

FIG. 6B is a digital image of the electrophoresed Bis-Tris gel shown in FIG. in 6A (imaged following a 45-second UV activation period) comprising 2-chloroethanol (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged with the Bis-Tris gel shown in FIG. 6F using an auto (optimal) exposure time of 17.308 seconds.

FIG. 6C is a digital image of the electrophoresed Bis-Tris gel shown in FIG. 6B (imaged following an additional 5-minute UV activation period) comprising 2-chloroethanol (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged with the Bis-Tris gel shown in FIG. 6G using an auto (optimal) exposure time of 5.385 seconds.

FIG. 6D is a digital image of a PVDF membrane (with no further UV activation) after the transfer of a Bis-Tris gel (shown in FIG. 6C) comprising 2-chloroethanol (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg each protein) in duplicate (lanes 1-3 and 9-11), and (iii) 10 μL Mark12 Unstained Standard (lanes 8 and 12); and imaged with the membrane shown in FIG. 6H using an auto (optimal) exposure time of 2.894 seconds.

FIG. 6E is a digital image of an electrophoresed Bis-Tris gel (with no UV activation period) comprising TCE (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged using a manual exposure time of 0.5 seconds.

FIG. 6F is a digital image of the electrophoresed Bis-Tris gel shown in FIG. 6E (imaged following a 45-second UV activation period) comprising TCE (0.05%); loaded with (i) E. coli lysate (lanes 4-7) at four different load amounts (2.5 μg, 5 μg, 10 μg, and 20 μg) (arrows indicating a fluorescent signal in the circled area), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged with the Bis-Tris gel shown in FIG. 6B using an auto (optimal) exposure time of 17.308 seconds.

FIG. 6G is a digital image of the electrophoresed Bis-Tris gel shown if FIG. 6F (imaged following an additional 5-minute activation period) comprising TCE (0.05%); loaded with (i) E. coli lysate (lanes 4-7) at four different load amounts (2.5 μg, 5 μg, 10 μg, and 20 μg) (arrows indicating a fluorescent signal in the circled area), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged with the Bis-Tris gel shown in FIG. 6C using an auto (optimal) exposure time of 5.385 seconds.

FIG. 6H is a digital image of a PVDF membrane (with no further activation) after the transfer of a Bis-Tris gel (shown in FIG. 6G) comprising TCE (0.05%); loaded with (i) E. coli lysate (lanes 4-7) at four different load amounts (2.5 μg, 5 μg, 10 μg, and 20 μg) (arrows indicating a fluorescent signal in the circled area), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg each protein) in duplicate (lanes 1-3 and 9-11) (arrows indicating a fluorescent signal in the dashed area), and (iii) 10 μL Mark12 Unstained Standard (lanes 8 and 12); and imaged with the membrane shown in FIG. 6D using an auto (optimal) exposure time of 2.894 seconds.

FIG. 6I is a digital image of an electrophoresed Bis-Tris gel (with no UV activation period) comprising 4,6-dichloro-5-fluoropyrimidine (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged using a manual exposure time of 0.5 seconds.

FIG. 6J is a digital image of the electrophoresed Bis-Tris gel shown in FIG. 6I (imaged following a 45-second activation period) comprising 4,6-dichloro-5-fluoropyrimidine (0.05%); loaded with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11) (arrows indicating a fluorescent signal in the circled area), and (iii) 10 μL Mark12 Unstained Standard (lanes 8 and 12); and imaged using an auto (optimal) exposure time of 5.114 seconds.

FIG. 6K is a digital image of the electrophoresed Bis-Tris gel shown in FIG. 6J (imaged following an additional 5-minute UV activation period) comprising 4,6-dichloro-5-fluoropyrimidine (0.05%); loaded with (i) E. coli lysate (lanes 4-7) (arrows indicating a fluorescent signal in the circled area) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged using an auto (optimal) exposure time of 2.097 seconds.

FIG. 6L is a digital image of a PVDF membrane (with no further UV activation) after transfer of a Bis-Tris gel (that shown in FIG. 6K) comprising 4,6-dichloro-5-fluoropyrimidine (0.05%); loaded with (i) E. coli lysate (lanes 4-7) (arrows indicating a fluorescent signal in the circled area) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg of each protein) in duplicate (lanes 1-3 and 9-11), and (iii) 10 μL Mark12 Unstained Standard (lanes 8 and 12); and imaged using an auto (optimal) exposure time of 0.664 seconds.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is P00698 (Gallus gallus lysozyme) demonstrating the six tryptophan residues at W1, W2, W3, W4, W5, and W6.

SEQ ID NO: 2 is P00698 (Gallus gallus lysozyme) demonstrating four modified tryptophan residues (W1, W2, W3, and W6) of the six tryptophan residues.

SEQ ID NO: 3 is a peptide fragment of Gallus gallus lysozyme having a sequence of GTDVQAWIR (unmodified fragment comprising W6).

SEQ ID NO: 4 is a peptide fragment of Gallus gallus lysozyme having a sequence of GTDVQAXIR (modified fragment, acyl 5).

SEQ ID NO: 5 is a peptide fragment of Gallus gallus lysozyme having a sequence of GYSLGNWVCAAK (unmodified fragment comprising W1).

SEQ ID NO: 6 is a peptide fragment of Gallus gallus lysozyme having a sequence of GYSLGNXVCAAK (modified, acyl 5).

SEQ ID NO: 7 is a peptide fragment of Gallus gallus lysozyme having a sequence of GYSLGNXVCAAK (modified, acyl 3).

SEQ ID NO: 8 is a peptide fragment of Gallus gallus lysozyme having a sequence of GYSLGNXVCAAK (modified, acyl acid).

SEQ ID NO: 9 is a peptide fragment of Gallus gallus lysozyme having a sequence of NTDGSTDYGILQINSRWWCNDGR (unmodified fragment comprising W2 and W3).

SEQ ID NO: 10 is a peptide fragment of Gallus gallus lysozyme having a sequence of 10 NTDGSTDYGILQINSRXXCNDGR (modified, acyl acid).

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

The methods described herein should not be construed as limiting in any way. Instead, the present disclosure is directed toward all novel and non-obvious features and aspects of the present disclosure, alone and in various combinations and sub-combinations with one another. The disclosed methods are not limited to any specific aspect or feature or combinations thereof, nor do the disclosed methods require that any one or more specific advantages be present, or problems be solved. Any theories of operation are to facilitate explanation, but the methods are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show all of the various ways in which the disclosed devices and methods can be used in conjunction with other devices and methods. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art. Furthermore, examples may be described with reference to directions indicated as “above,” “below,” “upper,” “lower,” and the like. These terms are used for convenient description, but do not imply any particular spatial orientation unless so indicated.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs.

Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximations unless the word “about” is recited. Furthermore, not all alternatives recited herein are equivalents.

A dashed bond (i.e., - - - ) as used in certain formulas described herein indicates an “optional” bond to a substituent or atom of the formula other than hydrogen in the sense that the bond (and in some embodiments, the substituent) may or may not be present. In heterocyclic compound formulas provided herein, the dashed bond is used to show where double bonds can be present for certain compounds but need not be in all compounds. The symbol “ [symbol] ” is used to indicate a bond disconnection in abbreviated structures/formulas provided herein.

To facilitate review of the various aspects of the present disclosure, the following explanations of specific terms are provided:

Aldehyde: A chemical functional group having a structure

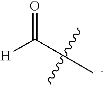 [SMILES: *C=O]

Aliphatic: A hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms. For example, from one to fifteen, from one to ten, from one to six, or from one to four carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a C═C double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, heteroaliphatic, heteroaryl, heterocycloaliphatic, hydroxyl, oxo, sulfonamide, sulfhydryl, thioalkoxy, or other functionality.

Alkoxy: A chemical functional group —OR, where R is a substituted or unsubstituted alkyl or a substituted or unsubstituted cycloalkyl group. In a substituted alkoxy, R is substituted alkyl or substituted cycloalkyl.

Aromatic: A cyclic, conjugated group or moiety of, unless specified otherwise, from 5 to 15 ring atoms having a single ring (e.g., phenyl, pyridinyl, or pyrazolyl) or multiple condensed rings in which at least one ring is aromatic (e.g., naphthyl, indolyl, or pyrazolopyridinyl), that is at least one ring, and optionally multiple condensed rings, have a continuous, delocalized n-electron system. Typically, the number of out of plane n-electrons corresponds to the Hückel rule (4n+2). The point of attachment to the parent structure typically is through an aromatic portion of the condensed ring system. For example,

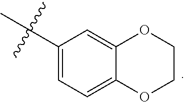 [SMILES: *c1ccc2c(c1)OCCO2]

However, in certain examples, context or express disclosure may indicate that the point of attachment is through a non-aromatic portion of the condensed ring system. For example,

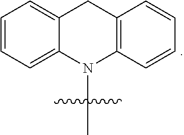 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety. Unless otherwise stated, an aromatic group may be substituted or unsubstituted.

Aryl: A monovalent aromatic carbocyclic group of, unless specified otherwise, from 6 to 15 carbon atoms having a single ring or multiple condensed rings in which at least one ring is aromatic, provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic, or tetracyclic.

Amido: A chemical functional group —C(O)N(R)(R) where R and R′ are independently hydrogen, alkyl, heteroalkyl, aliphatic, heteroaliphatic, aryl (such as optionally substituted phenyl or benzyl), heteroaryl, alkylsulfano, or other functionality.

Amino: A chemical functional group —N(R′)(R″) where R′ and R″ independently are selected from hydrogen, aliphatic, heteroaliphatic, aromatic, or any combination thereof.

Carbonyl: A chemical functional group with formula R—C(O)—R′, where R and R′ are a functional group or functionality and may be the same or different.

Carboxyl: A chemical functional group with formula —COOR where R is functional group or functionality.

Cyano: A chemical functional group

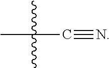 [SMILES: *C#N]

Energy Source: Electromagnetic radiation having a wavelength of 200 nm to 700 nm. For example, the energy source can be visible light having a wavelength of 400 nm to 700 nm. In another example, the energy source can be UV (ultraviolet) light having a wavelength of from 200 nm to 400 nm via transillumination or epi-illumination.

Ester: A chemical functional group

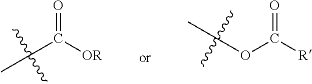

where R is aliphatic, heteroaliphatic, aromatic, or any combination thereof; and where R′ is hydrogen, aliphatic, heteroaliphatic, aromatic, or any combination thereof.

Ether: A chemical functional group —[R]ₙ—O—R′ where R′ and R″ independently are aliphatic, heteroaliphatic, aromatic, or any combination thereof; and n is 1 or 0.

Functional group: A specific group of atoms within a molecule that is responsible for the characteristic chemical reactions of the molecule. Exemplary functional groups include, without limitation, alkyl, alkenyl, alkynyl, aryl, epoxide, hydroxyl, carbonyl (ketone), aldehyde, carbonate ester, carboxylate, carboxyl, ether, ester, peroxy, hydroperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

Heteroaliphatic: An aliphatic compound or group having at least one carbon atom in the chain and at least one heteroatom, typically nitrogen, oxygen, boron, phosphorus, selenium, silicon, or sulfur. Heteroaliphatic compounds or groups may be substituted or unsubstituted, branched or unbranched, chiral or achiral, and/or acyclic or cyclic, such as a cycloheteroaliphatic group. In independent aspects of the disclosure, a heteroaliphatic group does not comprise a halogen atom.

Heteroaryl: An aromatic compound or group having at least one heteroatom, i.e., one or more carbon atoms in the ring has been replaced with an atom having at least one lone pair of electrons, typically nitrogen, oxygen, boron, phosphorus, selenium, silicon, or sulfur.

Heterocyclic Compound: A cyclic compound comprising at least one ring atom that is a heteroatom. Heterocyclic compounds used in compositions described herein can have a structure according to Formula I provided herein and can include heterocycles having no saturation, heterocycles having one or more sites of unsaturation, and/or aromatic heterocycles.

Hydroxyl: A chemical functional group —OH.

Isocyanate: A chemical functional group.

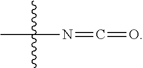 [SMILES: *N=C=O]

Ketone: A chemical functional group

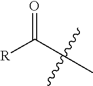

where R is other than hydrogen, such as aliphatic, heteroaliphatic, aromatic, or any combination thereof.

Medium: A material for providing a heterocyclic compound, wherein the providing can be in the form of being bound to or associated with the heterocyclic compound. In some aspects of the disclosure, the medium can be a solvent that can be associated with (e.g., mixed) a heterocyclic compound. In yet other aspects, the medium can be a solid or a gel that can be associated with (e.g., mixed) or bound to (e.g., covalently or non-covalently) the heterocyclic compound.

Nitrile: A chemical functional group

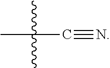 [SMILES: *C#N]

Nitro: A chemical functional group

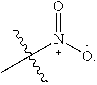

Oxime: A chemical functional group

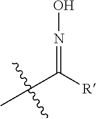

where R′ is a functional group.

Polypeptide: A polymer in which the monomers are amino acid residues that are joined together through amide bonds. The terms “polypeptide” or “protein” as used herein are intended to encompass any amino acid sequence and include modified sequences. The term “polypeptide” is specifically intended to cover naturally occurring proteins, as well as those which are recombinantly or synthetically produced. The term “residue” or“amino acid residue” includes reference to an amino acid that is incorporated into a protein, polypeptide, or peptide.

Sample: Any liquid, semi-solid or solid substance (or material) in or on which a target can be present. In particular, a sample can be a biological sample or a sample obtained from a biological material. A biological sample is any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease). A biological sample can also be a sample obtained from any organ or tissue (including a biopsy or autopsy specimen, such as a tumor biopsy) or can include a cell (whether a primary cell or cultured cell) or medium conditioned by any cell, tissue, or organ. In some examples, a biological sample is a nuclear extract. In some examples, a biological sample is bacterial cytoplasm. In other examples, a sample is a test sample. For example, a test sample is a cell, a tissue or cell pellet section prepared from a biological sample obtained from a subject. In an example, the subject is one that is at risk or has acquired a particular condition or disease.

Western Blot: An analytical technique used to detect specific proteins in a sample. The technique can use gel electrophoresis or other suitable procedures to separate proteins by size, shape, length, charge, or other characteristics. The proteins can then be transferred to a membrane and detected with binding agents that can be specific to one or more target proteins.

### II. Introduction

Current protein visualization methods do not allow for the rapid detection of proteins without excessive handling and result in decreased efficiency of high-throughput detection. For example, SDS-PAGE gel staining methods can take several hours to stain and de-stain and involve heating, require staining and de-staining reagents, and produce strong odors. Therefore, rapid and sensitive protein visualization methods are desired, which will increase the efficiency of high-throughput detection; allow for the rapid detection of protein without excessive handing that do not damage the medium, such as, but not limited to gels; and do not generate background signal.

Stain-free, in-gel protein labeling technology enables the visualization of SDS-PAGE gels with higher sensitivity and a preferable dynamic range for protein quantitation than traditional methods, which require staining and de-staining reagents. Disclosed herein is composition comprising a medium and a heterocyclic compound that reacts with proteins comprising an aromatic residue directly in the medium when exposed to an energy source and thereby fluorescently activate the proteins, which can then be imaged. Moreover, the heterocyclic compounds disclosed herein are not fluorescent and thus can be distributed uniformly throughout the medium without substantially increasing background. However, when the heterocyclic compound associates with an aromatic residue of an amino acid and is excited by an energy source, light is emitted with a peak in the visible spectrum and thus is fluorescently activated. Furthermore, these protein modifications are minimal and do not affect protein transfer or downstream antibody binding in western blotting.

The present disclosure allows for in-medium detection of proteins in a sample using heterocyclic compounds using chemistry that is non-halogenated, safer, and environmentally friendly. Aromatic amino acid residues in proteins react with the heterocyclic compounds to produce an amino acid adduct upon the exposure of an energy source, which results in red-shifted fluorescence that can be readily imaged. Furthermore, the compositions disclosed herein can be distributed in a medium to react with proteins in a sample. For example, compositions disclosed herein can be distributed in a medium such as, but not limited to, a gel, and can react with proteins in a sample by providing an electric charge on the compound and incorporating the charged compound in one or both electrode buffers in an electrophoresis system, when a biological sample is loaded onto the gel and the electrodes that are immersed in the buffers are energized to appropriate polarities to cause electrophoretic separation of the proteins in the sample to occur, wherein the heterocyclic compound will migrate into and thorough the gel by virtue of its charge and thus utilizing the electrophoretic principle to transfer the compound from the electrode buffer into the gel. The penetration of the gel with the heterocyclic compounds will occur concurrently with the reparatory migration of the proteins with the gel, avoiding any need for pre-treatment of the sample or gel or for post-treatment of the gel.

### III. Composition

Disclosed herein are compositions comprising a heterocyclic compound comprising a cyclic heteroatom that can be used to detect proteins in a sample. Such heterocyclic compounds can associate with aromatic amino acid residues of a biomolecule to produce a conjugate upon association. In some aspects of the disclosure, the association takes places between the cyclic heteroatom and an aromatic ring of the aromatic amino acid residue of the biomolecule. In some aspects of the disclosure, the compositions disclosed herein may further comprise a medium for providing the heterocyclic compound. In certain aspects, the medium can be a solvent. In other aspects disclosed herein, the medium can be a solid or a gel.

In some aspects of the disclosure, the medium can be one or more solvents that can dissolve the heterocyclic compound such as, but not limited to, an aqueous solvent, or an alcohol. Exemplary alcohols can include, but are not limited to methanol, ethanol, isopropanol, or any combination thereof. In certain aspects, the solvent can be a buffer such as, but not limited to, Bis-Tris, Tris-glycine, Tris-acetate, and/or tricine. In aspects disclosed herein, the solvent can be a protein separation matrix.

In particular aspects disclosed herein, the medium can be a gel. In certain aspects, the gel can be, but is not limited to, a polyacrylamide gel, a starch gel, or an agarose gel. In one example, the medium is a precast polyacrylamide gel comprising the heterocyclic compound for purposes of protein separation by electrophoresis. In another example, the heterocyclic compound can be deposited onto the surface of a polyacrylamide gel by soaking the polyacrylamide gel in a solution comprising the heterocyclic compound.

In some aspects of the disclosure, the medium can be a membrane such as, but not limited to, a membrane used in western blots. In aspects disclosed herein, a gel can be transferred onto a membrane via wet, semi-dry, or dry transfer. In certain aspects, the membrane can be, but is not limited tom a nitrocellulose membrane, polyvinylidene difluoride (PVDF) membrane, or a nylon membrane.

In some aspects of the disclosure, the heterocyclic compound can be deposited and/or adhered to a solid surface. In certain aspects, the solid surface can be, but is not limited to a glass surface or plastic surface, such as a surface of a tube, cuvette, microplate, cassette, well, slide, test strip, and the like.

Disclosed herein are compositions comprising a heterocyclic compound that can be used to detect a biomolecule by generating a fluorescent signal after associating with the biomolecule. Such compositions can produce a conjugate upon association with an aromatic amino acid residue of the biomolecule to produce a fluorescent signal. In certain aspects, the heterocyclic compound is typically selected to associate with an aromatic amino acid residue of the biomolecule and thus produce a conjugate upon association with the aromatic amino acid residue of the biomolecule, the association taking place between the cyclic heteroatom group and an aromatic ring of the aromatic amino acid residue of the biomolecule.

In some aspects of the disclosure, the heterocyclic compound comprise at least one heteroatom selected from N, O, P, S, B, or Se that is present within the heterocycle of the compound. In certain aspects, the heterocyclic compound has a structure according to Formula I, or is an enantiomer, diastereomer, tautomer, salt, solvate, and/or isotopically substituted derivative thereof.

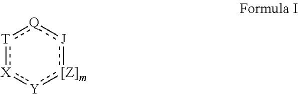

With reference to Formula I, each of J, Q, T, X, Y, and Z is the same or different, and each of J, Q, T, X, Y, and Z independently is selected from oxygen (O); phosphorus (P); boron (B); nitrogen (N); sulfur (S); selenium (Se); N(R″) or N⁺[R″], wherein R″ independently is selected from hydrogen, aliphatic, cycloaliphatic, cycloheteroaliphatic, or aryl; or BR^{c} or C(R^{c})ₙ, wherein n is 1 or 2; provided that at least one of J, Q, T, X, Y, and Z is O, P, N, S, Se, N(R″), N⁺[R′], or BR^{c}; and m is zero is or one. In some aspects of the disclosure, each R^{c} independently is selected from hydrogen, halo, aliphatic, heteroaliphatic, ether, amino, carbonyl, carboxyl, ketone, aldehyde, isocyanate, cyano, oxime, nitro, nitrile, a salt thereof, or an anionic form thereof. In certain aspects, when one or more of J, Q, T, X, Y, and Z are CR^{c}, two R^{c} groups, together with the carbon atoms to which they are attached, can form an aliphatic ring system, a heteroaliphatic ring system, or an aromatic ring system.

In independent aspects, compositions disclosed herein do not include haloaliphatic compounds and/or haloaliphatic-substituted heterocyclic compounds. In such independent aspects, haloaliphatic compounds and/or haloaliphatic-containing substituents can include trihaloaliphatic alcohols, trihaloaliphatic acids, trihaloaliphatic amines, and/or trihaloaliphatic alkanes. For example, chloroform, trichloroacetic acid, and/or trichloroethanol. Examples included herein that use such compounds are used solely as comparative compounds and are not intended to be included within the scope of the present disclosure.

In some aspects of the disclosure, with reference to Formula I, when m is zero and J and Y are bound by a single or double bond, the heterocyclic compound can have a structure according to Formula II.

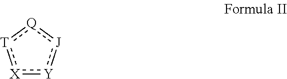

In particular aspects disclosed herein, with reference to Formula II, at least one of J, Q, T, X, or Y is S, Se, O, N, N(R′), N⁺[R″], or BR^{c}, and one or more of the remaining J, Q, T, X, or Y groups is CR^{c}, wherein the R^{c} group is hydrogen, halo, or cyano. In certain aspects, one of J, Q, T, X, or Y is N or N(R″), wherein R″ is hydrogen; one of J, Q, T, X, or Y is BR^{c}; one of J, Q, T, X, or Y is O; and the remaining J, Q, T, X, or Y groups are CR^{c}. In yet other aspects, one of J, Q, T, X, or Y is N or NR″, wherein R″ is hydrogen; one of J, Q, T, X, or Y is BR^{c}; and the remaining J, Q, T, X, or Y groups are CR^{c}. In yet other aspects, one of J, Q, T, X, or Y is N or NR″, wherein R″ is hydrogen; one of J, Q, T, X, or Y is Se; and the remaining J, Q, T, X, or Y groups are CR^{c}. Solely by way of example, the heterocyclic compound can be selected from the compounds listed below.

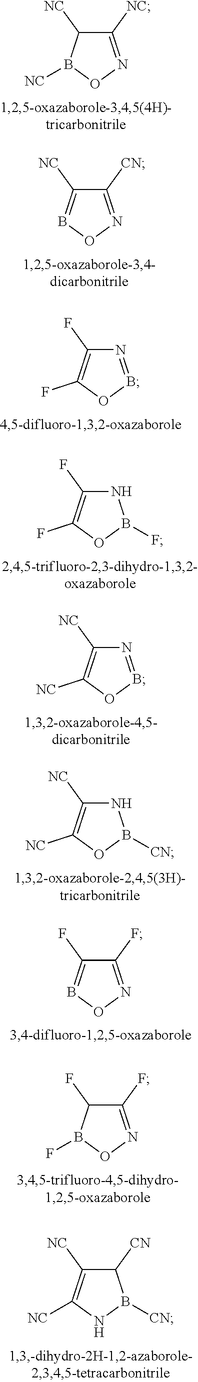

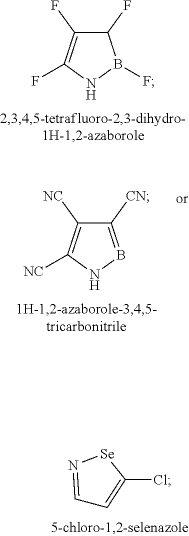 [SMILES: FB1NC(F)=C(F)C1F ; N#Cc1b[nH]c(C#N)c1C#N ; Clc1ccn[se]1]

In some aspects of the disclosure, with reference to Formula II, two of J, Q, T, X, or Y are N, N(R″), N⁺[R″], or a combination thereof, wherein R″ is hydrogen; and the remaining J, Q, T, X, or Y groups are (i) B; (ii) BR^{c}; (iii) O; (iv) Se; and/or (v) CR^{c}, wherein R^{c} is hydrogen, cyano, or halo. In certain aspects, one of J, Q, T, X, or Y is B or BR^{c}; one of J, Q, T, X, or Y is O; two of J, Q, T, X, or Y are N, and the remaining J, Q, T, X, or Y is CR^{c}, wherein R^{c} is hydrogen, halo, or cyano. In certain aspects, one of J, Q, T, X, or Y is B or BR^{c}; two of J, Q, T, X, or Y are N or NR″, and the remaining J, Q, T, X, or Y groups are CR^{c}, wherein R^{c} is hydrogen, halo, or cyano. Solely by way of example, the heterocyclic compound can be selected from the compounds listed below.

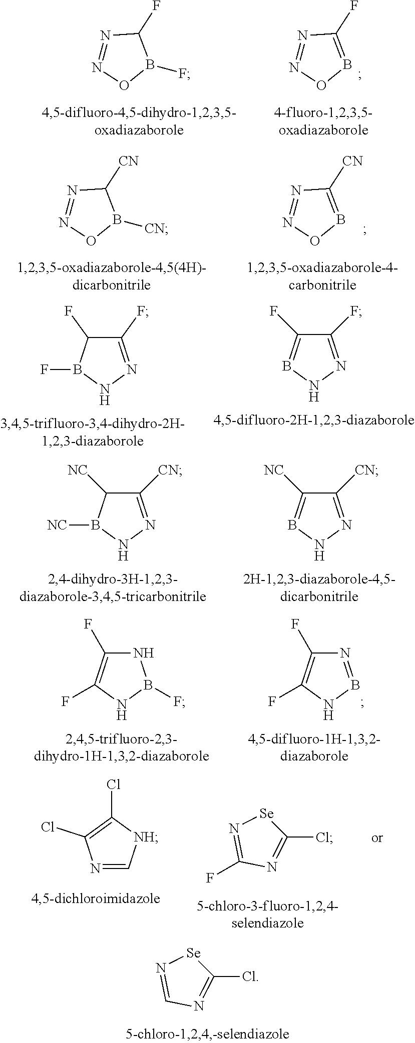

In some aspects of the disclosure, with reference to Formula II, two of J, Q, T, X, or Y are independently (i) S and (ii) B or BR^{c}. In certain aspects, three of J, Q, T, X, or Y independently are (i) N or NH; (ii) B or BR^{c}; and (iii) S, with the remaining J, Q, T, X, or Y being C(R^{c})ₙ, wherein n is 1 or 2, and R^{c} is hydrogen, halo, and/or cyano. Solely by way of example, the heterocyclic compound can be selected from the compounds listed below.

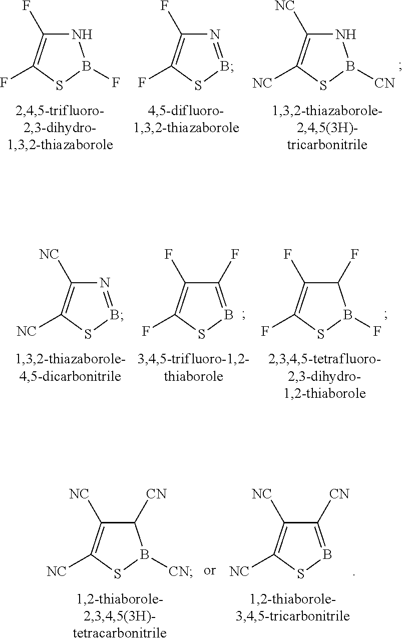

In particular aspects disclosed herein, with reference to Formula II, one or more of J, Q, T, X, or Y is nitrogen; one or more of J, Q, T, X, or Y is N(R″) wherein the R″ group is hydrogen, aliphatic, or cycloaliphatic; and at least two of J, Q, T, X, or Y individually is CR^{c}, wherein the two R^{c} groups of the two CR^{c} groups, together with the carbon atoms to which they are bound, form an aryl or heteroaryl ring system. In some aspects of the disclosure, the aryl or heteroaryl ring system may comprise a halo substituent. Solely by way of example, the heterocyclic compound can be selected from the compounds shown below.

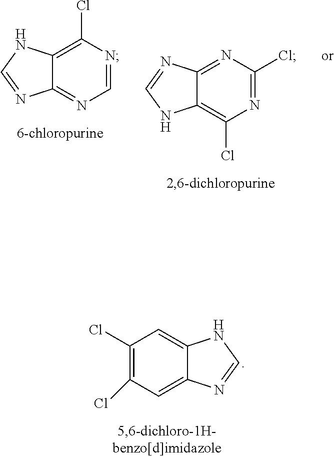

In particular aspects disclosed herein, with reference to Formula I, when m is 1, the heterocyclic compound can have a structure according to Formula III

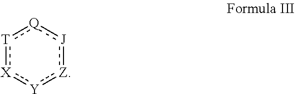

With reference to Formula III, each of Q, T, X, Y, and Z are the same or different, and each of Q, T, X, Y, and Z independently is selected from oxygen (O); phosphorus (P); boron (B); nitrogen (N); sulfur (S); selenium (Se); N(R″) or N⁺[R″], wherein R″ independently is selected from hydrogen, aliphatic, cycloaliphatic, cycloheteroaliphatic, or aryl; or BR^{c} or CR^{c}, wherein (i) each R^{c} independently is selected from hydrogen, aliphatic, heteroaliphatic, halo, ether, amino, carbonyl, carboxyl, ketone, aldehyde, isocyanate, cyano, oxime, nitro, nitrile, a salt thereof, or an anionic form thereof, or (ii) for CR^{c}, two R^{c} groups, together with the carbon atoms to which they are attached, can form an aliphatic ring system, a heteroaliphatic ring system, or an aromatic ring system; provided that at least one of Q, T, X, Y, and Z is O, P, N, S, Se, N(R″), N⁺[R″], or BR^{c}.

In some aspects of the disclosure, with reference to Formula III, at least one of J, Q, T, X, Y, or Z is N, N(R″), or N⁺[R″] and at least one of J, Q, T, X, Y, or Z is B or BR^{c}, wherein the R^{c} group is halo or cyano. Solely by way of example, the heterocyclic compound can be selected from the compounds shown below.

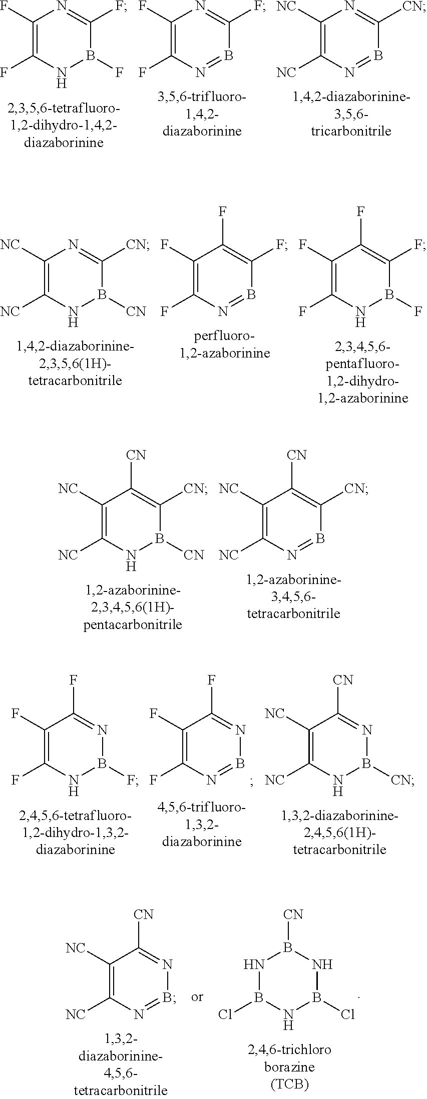

In aspects disclosed herein, with reference to Formula III, at least one of J, Q, T, X, Y, or Z is N or N⁺[R″] and at least two of J, Q, T, X, Y, or Z are CR^{c}, wherein each R^{c} group independently is selected from halo, heteroaryl, carbonyl, nitro, nitrile, carboxyl, isocyanate, oxime, amino, alkoxy, or aralkyl. In certain aspects, at least two of J, Q, T, X, Y, or Z are CR^{c} wherein each R^{c} group is halo. Solely by way of example, the heterocyclic compound can be selected from the compounds shown below.

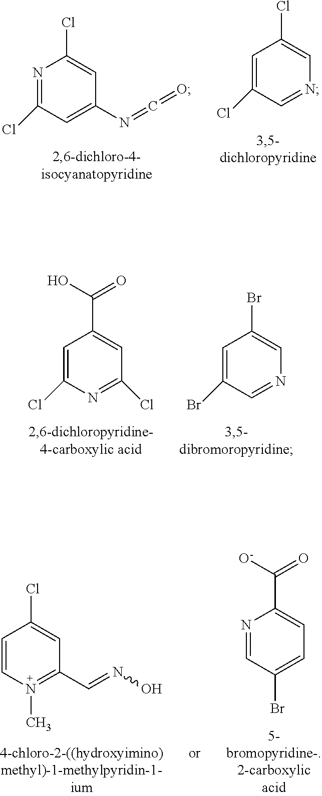

In some aspects of the disclosure, with reference to Formula III, at least two of J, Q, T, X, Y, or Z are N. In yet additional aspects, at least one of J, Q, T, X, Y, or Z is CR^{c}, wherein R^{c} is halo. In certain aspects, two of J, Q, T, X, Y, or Z are CR^{c}, wherein each R^{c} group is halo. In aspects disclosed herein, J, Q, T, X, Y, or Z are selected such that two of J, Q, T, X, Y, or Z are nitrogen separated by one CR^{c} group. In some such aspects of the disclosure, the R^{c} group is hydrogen. Solely by way of example, the heterocyclic compound can be selected from the compounds shown below.

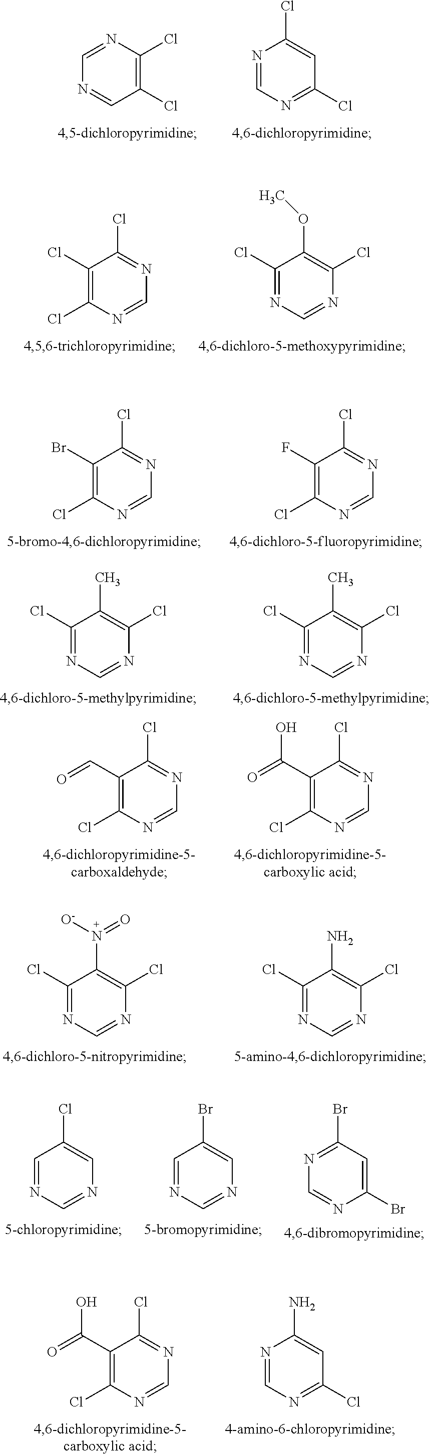

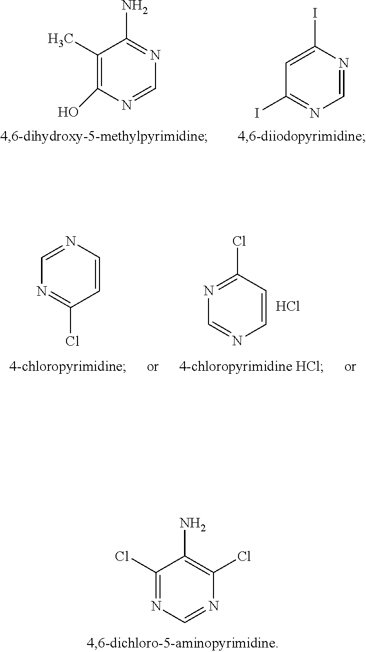

In some aspects of the disclosure, with reference to Formula III, at least two of J, Q, T, X, Y, or Z are N. In certain aspects, at least one of J, Q, T, X, Y, or Z is CR^{c}; wherein R^{c} is halo. In aspects disclosed herein, at least two of J, Q, T, X, Y, or Z are CR^{c}; wherein the two R groups are halo. In some aspects of the disclosure, J, Q, T, X, Y, or Z are selected such that two of J, Q, T, X, Y, or Z are nitrogen separated by one J, Q, T, X, Y, or Z that is CR^{c}. In such aspects, R^{c} is other than hydrogen. Solely by way of example, the heterocyclic compound can be selected from the compounds shown below.

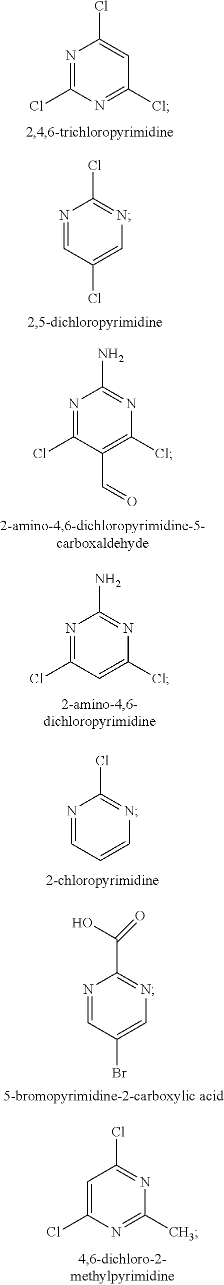

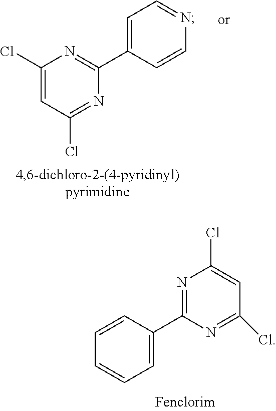 [SMILES: Clc1cc(Cl)nc(-c2ccncc2)n1 ; Clc1cc(Cl)nc(-c2ccccc2)n1]

In some aspects of the disclosure, with reference to Formula III, J, Q, T, X, Y, or Z are selected such that two of J, Q, T, X, Y, or Z are nitrogen separated by two CR^{c} groups. Solely by way of example, the heterocyclic compound can be selected from the compounds shown below.

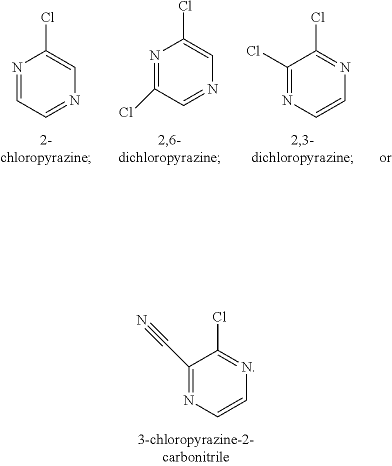

The composition disclosed herein may comprise a heterocyclic compound concentration that ranges from 0.001% to 10% by volume or by weight. In some aspects of the disclosure, the composition disclosed herein can have a heterocyclic compound concentration that ranges from 0.001% to 5% by volume or by weight. In preferable aspects, the composition disclosed herein can have a heterocyclic compound concentration that ranges from 0.001% to 3% by volume or by weight, such as from 0.001% to 2.5% by volume or by weight, 0.001% to 2.0% by volume or by weight, 0.001% to 1.5% by volume or by weight, from 0.001% to 1.25% by volume or by weight, 0.001% to 1.0% by volume or by weight, 0.001% to 0.5% by volume or by weight, from 0.001% to 0.05% by volume or by weight.

In some aspects of the disclosure, the composition may further comprise a buffer. In particular aspects disclosed herein, the buffer can be Tris-glycine, Bis-Tris, Tris-Acetate, Tris-Tricine IEF, Zymogram, or any combination thereof. In some aspects of the disclosure the buffer can be low Tris-SDS buffer (Tris HCl (63 mM), glycerol (10%), SDS (2%), bromophenol blue (0.0025%), pH 6.8); Tris-glycine SDS buffer (Tris base (25 mM), glycine (192 mM), SDS (0.1%), pH 8.3); Tris native buffer (Tris HCl (100 mM), glycerol (10%), bromophenol blue (0.00025%), pH 8.6); Tris glycine native buffer (Tris base (25 mM), glycine (192 mM), pH 8.3); Tris LDS buffer (Tris base (141 mM), Tris HCl (106 mM), LDS (2%), EDTA (0.51 mM), SERVA Blue G-250 (0.22 mM), phenol red (0.175 mM), pH 8.5); Bis-Tris MES SDS buffer (MES (50 mM), Tris base (50 mM), SDS (0.1%), EDTA (1 mM), pH 7.3); Tris MOPS SDS buffer (MOPS (50 mM), Tris base (50 mM), SDS (0.1%), EDTA (1 mM), pH 7.7); low Tris Tricine SDS buffer (Tris base (50 mM), Tricine (50 mM), SDS (0.1%), pH 8.24); high Tris SDS buffer (Tris HCl (450 mM), glycerol (12/), SDS (4%), Coomassie Blue G (0.00075%), phenol red (0.0025%), pH 8.45); high Tris-Tricine SDS buffer (Tris base (100 mM), Tricine (100 mM), SDS (0.1%), pH 8.3); IEF buffer, pH 3-7 (Lysine (40 mM), glycerol (15%); IEF cathode buffer, pH 3-7 (Lysine (40 mM)); IEF cathode buffer, pH 3-10 (arginine (20 mM), lysine (20 mM)); IEF anode buffer (phosphoric acid 85% (7 mM)); Zymogram Tris SDS buffer (Tris HCl (63 mM), glycerol (10%), SDS (2%), bromophenol blue (0.0025%), pH 6.8); Zymogram Tris-glycine SDS buffer (Tris base (25 mM), glycine (192 mM), SDS (0.1%), pH 8.3), or any combinations thereof.

### IV. Method of Making

Also disclosed herein are aspects of a method for making the composition disclosed herein. In some aspects of the disclosure, the medium and heterocyclic compound can be added to a container simultaneously or sequentially, in any order. In a particular disclosed aspect, a heterocyclic compound disclosed herein and a solvent comprising a buffer, a protein separation matrix, or a combination thereof can be added to a container to make the composition disclosed herein. For example, the method of making may comprise making a hand cast solution by adding the heterocyclic compound to a container comprising an aqueous solvent or an alcohol (e.g., methanol, ethanol, propanol, or any combination thereof). In another example, the method of making may comprise adding the heterocyclic compound to a container comprising a buffer.

In certain aspects, the method for making the composition disclosed herein can further comprise making a precast gel. In such aspects, the precast gel can be made by adding a monomer, crosslinker, a initiator, or any combination thereof to a container that may or may not comprise the heterocyclic compound disclosed herein. In aspects disclosed herein, the monomer can be a monomer (or combination of different monomers) that is capable of polymerizing to form a gel. In some aspects of the disclosure, the monomer can be acrylamide, the crosslinker can be bisacrylamide (N,N′-methylenediacrylamide), and the initiator can be ammonium persulfate (APS). In aspects disclosed herein, the composition may further comprise a promoter for catalyzing/promoting the polymerization reaction, such as but not limited to, N,N,N′,N′-tetramethylethylenediamine (TEMED), or a combination thereof to form a crosslinked polymer network as illustrated by Scheme I.

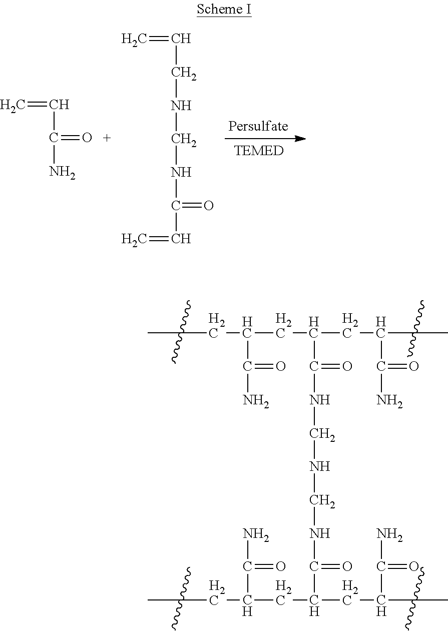

In certain aspects, the acrylamide and bisacrylamide can be used in amounts providing a ratio of 10:1 to 100:1 (acrylamide-bisacrylamide), preferably, 15:1 to 50:1 (acrylamide:bisacrylamide), and more preferably, 20:1 to 40:1 (acrylamide:bisacrylamide).

In some aspects of the disclosure, a polymerized gel can be formed inside a container (e.g., a cassette), wherein the heterocyclic compound can be deposited and/or adhered to the gel after it is formed. For example, the polymerized gel can be removed from the cassette and soaked in the composition disclosed herein that comprises a combination of the heterocyclic compound and a solvent. Alternatively, after removing the gel from the cassette, the heterocyclic compound disclosed herein can be deposited on the polymerized gel.

In other aspects disclosed herein, the heterocyclic compound can be deposited on and/or adhered to a solid surface. In certain aspects, the heterocyclic compound can be deposited onto the surface of a tube, cuvette, microplate, cassette, well, slide, test strip, and the like. In some aspects of the disclosure, a polymer coating can be formed on a solid surface (e.g., a polymer and/or glass surface) and used to attach the heterocyclic compound by, for example, a covalent bond, ionic interaction, hydrophilic interaction, hydrophobic interaction, affinity interaction, hydrogen bonding, and/or Van der Waals force interaction.

### V. Method of Using

The composition and kit disclosed herein can be used to detect the presence of one or more biomolecules in a sample by generating a detectable fluorescent signal upon association of the heterocyclic compound with an aromatic amino acid residue of the biomolecule, such as a tryptophan, tyrosine, and/or phenylalanine residue. Without being bound by a theory of operation, by exposing the amino acid conjugate to an energy source, the heterocyclic compound can react with an aromatic amino acid residue to form extended conjugated aromatic amino acid products and produce a fluorescent emission that can be detected and quantified. The composition disclosed herein can be used in place of a protein stain in gel electrophoresis and/or in protein quantification and normalization.

Without being bound to a single theory, it currently is believed that a reaction between a heterocyclic compound and an aromatic amino acid residue of a biomolecule can take place as illustrated in Scheme II. As shown in Scheme II, a heterocyclic compound (e.g., 4,6-dichloropyrimadine) can associate and form a conjugate with an aromatic amino acid (e.g., tryptophan) under activation by an energy source. Exemplary, but non-limiting, conjugate species are shown in Scheme II. These conjugates formed with aromatic amino acid residues of biomolecules produces a fluorescent signal, which can be detected using techniques described herein. Without being limited to a single theory concerning the mechanism involved, it currently is believed that the heterocyclic compounds can react with aromatic amino acid residues of biomolecules according to a mechanism as described in Scheme Ill.

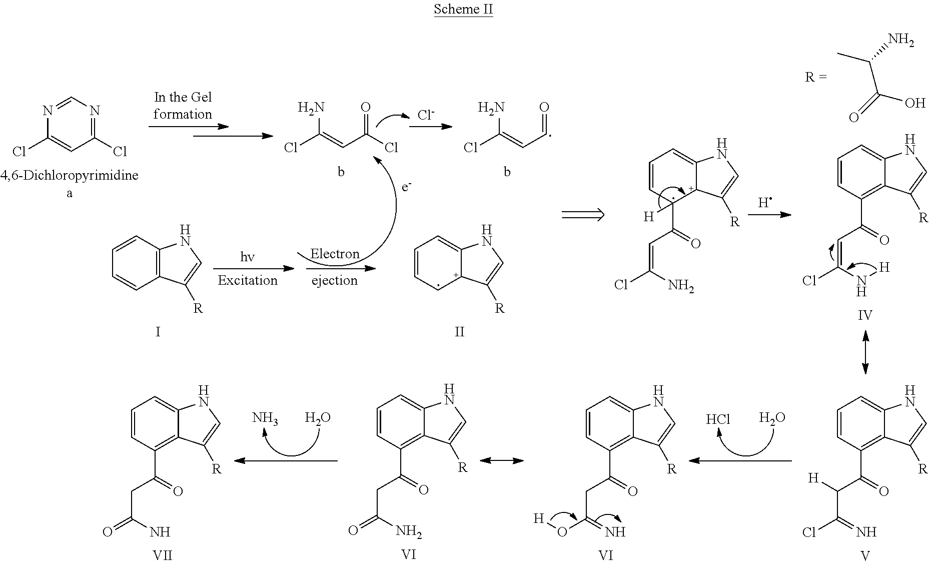

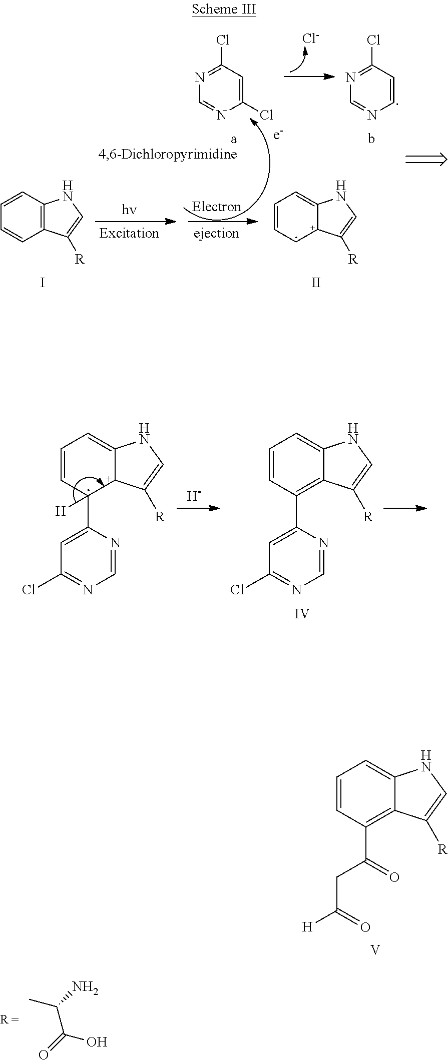

In certain aspects of the disclosure, the method may comprise quantitating one or more biomolecules by combining a sample comprising the one or more biomolecules with the heterocyclic compound disclosed herein and exposing the combination to an energy source (e.g., UV light) to form a conjugate with an aromatic amino acid and wherein a fluorescent signal is emitted. The conjugate can be imaged and/or scanned for fluorescence and compared to a reference standard. In some aspects of the disclosure, the combination of the sample and the heterocyclic compound can be formed in a container, which can be a tube, cuvette, microplate, well plate, and the like. In one example, the composition disclosed herein may comprise a gel and a heterocyclic compound, wherein the composition can be loaded with one or more samples that are spatially separated in different lanes for comparison. In another example, a western blot membrane comprising the composition disclosed herein may comprise multiple samples that are spatially separated at corresponding locations for migration during the western blot protocol.

In some aspects of the disclosure, the biomolecule can be a peptide, protein, or other biomolecule comprising an aromatic amino acid residue, such as a tryptophan, tyrosine, and/or phenylalanine residue. Using the disclosed method, the biomolecule becomes labeled with the heterocyclic compound. The labeled biomolecule (e.g., a labeled protein) can be prepared for polyacrylamide gel electrophoresis (PAGE), and/or imaged without further UV exposure. Biomolecule quantitation can be performed prior to, or after, electrophoresis.

In some aspects of the disclosure, the composition disclosed herein can be used in gel electrophoresis by adding a sample to a composition comprising a gel and a heterocyclic compound. After the sample is added, the gel can be electrophoresed and activated by exposing the gel to an energy source. After the gel is electrophoresed and activated, the method can further comprise imaging the gel to visualize the fluorescence produced by formation of the heterocyclic compound-biomolecule conjugate. In certain aspects, heterocyclic compounds that react with aromatic amino acid residues of the biomolecule can be distributed through a gel to react with the proteins in the gel by imposing an electric charge on the compounds and incorporating the charged compound in one or both electrode buffers in an electrophoresis system. When a biological sample is loaded onto the gel and the electrodes that are immersed in the buffers are energized to the appropriate polarities, electrophoretic separation of the protein will occur. Thus, the heterocyclic compound will migrate into and through the gel by virtue of its charge, thereby utilizing the electrophoretic principle to transfer the heterocyclic compound from the electrode buffer into the gel. The penetration of the gel with the heterocyclic compounds will occur concurrently with the reparatory migration of the proteins with the gel and thus pre-treatment of the sample or post-treatment of the gel is not required.

In some aspects of the present disclosure, a gel made according to a method described herein that comprises a heterocyclic compound according to the present disclosure can be transferred to a membrane by wet, semi-dry, or dry transfer. Membranes such as, but not limited to, nitrocellulose, polyvinylidene difluoride (PVDF), and/or nylon membrane can be used. Typically, in a wet transfer, the gel is equilibrated in a transfer buffer and placed in a “transfer sandwich” (e.g., a construct comprising a filter paper-gel-membrane-filter paper configuration), cushioned by pads, and pressed together by a support grid. The transfer sandwich is placed vertically in a tank between stainless steel/platinum wire electrodes and the tank is filled with a transfer buffer. One or more gels can be electro-transferred at constant current such as from 0.1 A to 2 A or voltage from 5 V to 300 V from 1 hour to 24 hours. If a higher current or voltage is used, the method can further comprise providing a cooling system to dissipate heat. In a semi-dry transfer, the transfer sandwich is placed horizontally between two electrode plates and the amount of buffer used in the transfer is selected to correspond to amounts contained in the transfer sandwich. Membrane and filter paper sheets are cut to the gel size without overhangs and the gel and filter paper are equilibrated with a transfer buffer. The transfer is performed at constant current such as, but not limited to, 0.1 A to 1 A or 10 V to 25 V for a suitable period of time, such as 10 minutes to 60 minutes. In a dry transfer, a transfer sandwich containing a gel matrix that incorporates buffer is used instead of buffer tanks or soaked filter paper.

Gel-to-membrane transfer efficiency can be assessed by adding a sample to a composition described herein comprising a solvent and a heterocyclic compound (e.g., a hand-cast solution).

Alternatively, gel-to-membrane transfer efficiency can be assessed by adding a sample to a composition described herein comprising a gel and a heterocyclic compound. The sample can then be electrophoresed and activated. After electrophoresis and activation, the gel can be imaged to visualize the presence of one or more aromatic amino acid-containing biomolecules using fluorescence and transferred to a membrane. After transfer, the sample can be imaged and compared to an image of the gel generated prior to transferring to the membrane.

In certain aspects, after the gel has been transferred to a membrane, the method may further comprise normalizing immunodetection results from blots (membranes) to total protein. In some aspects of the disclosure, the membrane is immunodetected by western blotting. Following the western blot, the immunodetected membrane is imaged to compare antibody-dependent signal to total protein signal.

In particular aspects disclosed herein, an energy source can be provided upon the association of the heterocyclic compounds disclosed herein with an aromatic amino acid residue of the biomolecule and activate the amino acid conjugate formed upon association. In some aspects of the disclosure, an energy source capable of producing electromagnetic radiation can be used to initiate forming conjugates as described above for detecting the presence of one or more biomolecules in a sample. In some aspects of the disclosure, the electromagnetic radiation can have a wavelength range from 200 nm to 700 nm. In some aspects of the disclosure, the energy source is capable of producing visible light having a wavelength range from 400 nm to 700 nm and/or UV (ultraviolet) light having a wavelength of from 200 nm to 400 nm.

In some aspects of the disclosure, the energy source can be any suitable energy source, such as but not limited to, a UV lamp, a transillumination device, and/or an epi-illumination device. In certain aspects, the fluorescent emission can be detected and quantified via a densitometer, photographic film, laser scanner, camera, photodiode, a charged-coupled device detector, complementary metal-oxide semiconductor detector, spectrophotometer, and the like.

In certain aspects, the energy source can be used to provide electromagnetic radiation for a time period ranging from greater than 0 milliseconds up to 1,800,000 milliseconds, preferably from 1 millisecond to 1,000,000 milliseconds, 600,000 milliseconds, and more preferably from 1 millisecond to 600,000 milliseconds.

### VI. Kit

The present disclosure also describes kits for implementing the methods discussed herein and/or kits that contain compositions discussed herein.

In certain aspects, the present disclosure describes a kit for detecting one or more biomolecules. The kit may comprise a medium, a heterocyclic compound, and instructions for using and/or making the medium and the heterocyclic compound.

In aspects disclosed herein, the medium can be a solvent that can be associated with (e.g., mixed) a heterocyclic compound. For example, the solvent can be a buffer, a protein separation matrix, an aqueous solvent, or an alcohol (e.g., methanol, ethanol, propanol, or any combination thereof).

In yet other aspects, the medium can be a solid or a gel that can be associated with (e.g., mixed) or bound to (e.g., covalently, or non-covalently) with the heterocyclic compound. For example, the medium can be, but is not limited to, a polyacrylamide gel, starch gels, or agarose gels. Alternatively, the gel can be membrane such as, but not limited to, nitrocellulose, polyvinylidene difluoride and nylon. In yet other aspects of the disclosure, the medium can be a solid, such as a solid substrate as described herein.

In some aspects of the disclosure, the kit may further comprise a container for making/and or using the medium and the heterocyclic compound. In particular aspects disclosed herein, the container can be a tube, cuvette, microplate, multi-well plate, a test strip, a disc, a cassette, and the like.

In particular aspects of the disclosure, the instructions can describe a method for making the medium and the heterocyclic compound. In certain aspects, the instructions can describe a method of making a precast gel from a hand-cast solution disclosed herein. In other aspects, the instructions can provide a method of making the hand-cast solution disclosed herein.

In some aspects of the disclosure, the instructions can further comprise information regarding using the composition and evaluating results obtained from using the composition. For example, in certain aspects of the disclosure, the instructions can provide a method of using the medium and heterocyclic compound. In some aspects of the disclosure, the instructions may comprise a method for quantitating one or more biomolecules discussed herein. In aspects disclosed herein, the instruction may comprise a method for using the medium and heterocyclic compound in gel electrophoresis. In particular aspects disclosed herein, the instructions may comprise a method for assessing the efficiency of transferring a gel to membrane discussed herein. In certain aspects, the instructions may provide a method for normalizing immunodetection results from blots (membranes) to total protein.

### VII. Overview of Several Aspects

Disclosed herein are aspects of a composition for detecting a biomolecule, comprising a medium; and a heterocyclic compound comprising at least one ring heteroatom selected from N, O, P, S, B, or Se; or an enantiomer, diastereomer, tautomer, salt, solvate, and/or isotopically substituted derivative thereof; wherein, the heterocyclic compound is selected to associate with an aromatic amino acid residue of the biomolecule and wherein the heterocyclic compound produces a conjugate upon association with the aromatic amino acid residue of the biomolecule, the association taking place between the cyclic heteroatom group and an aromatic ring of the aromatic amino acid residue of the biomolecule.

In some aspects of the present disclosure, the heterocyclic compound has a structure according to Formula I,

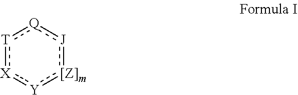

wherein: each of J, Q, T, X, Y, and Z independently is selected from O; P; B; N; S; Se; N(R″) or N⁺[R″], wherein R″ independently is selected from hydrogen, aliphatic, cycloaliphatic, cycloheteroaliphatic, or aryl; or BR^{c} or C(R^{c})ₙ, wherein n is 1 or 2, (i) each R^{c} independently is selected from hydrogen, halo, aliphatic, heteroaliphatic, ether, amino, carbonyl, carboxyl, ketone, aldehyde, isocyanate, cyano, oxime, nitro, nitrile, a salt thereof, or an anionic form thereof, or (ii) for CR^{c}, two R^{c} groups, together with the carbon atoms to which they are attached, form an aliphatic ring system, a heteroaliphatic ring system, or an aromatic ring system; provided that at least one of J, Q, T, X, Y, and Z is O, P, N, S, Se, N(R″), N⁺[R″], or BR^{c}; and m is zero is or one.

In any or all of the above aspects, where m is zero and J and Y are bound by a single or double bond, and the heterocyclic compound has a structure according to Formula II

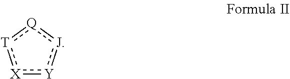

In any or all of the above aspects, at least one of J, Q, T, X, or Y is S, Se, O, N, N(R″), N⁺[R″], or BR^{c}, and one or more of the remaining J, Q, T, X, or Y groups is CR^{c}, wherein the R^{c} group is hydrogen, halo, or cyano.

In any or all of the above aspects (i) one of J, Q, T, X, or Y is N or N(R″), wherein R″ is hydrogen; one of J, Q, T, X, or Y is BR^{c}; one of J, Q, T, X, or Y is O; and the remaining J, Q, T, X, or Y groups are CR^{c}; (ii) one of J, Q, T, X, or Y is N or NR″, wherein R″ is hydrogen; one of J, Q, T, X, or Y is BR^{c}; and the remaining J, Q, T, X, or Y groups are CR^{c}; or (iii) one of J, Q, T, X, or Y is N or NR″, wherein R″ is hydrogen; one of J, Q, T, X, or Y is Se; and the remaining J, Q, T, X, or Y groups are CR^{c}.

In any or all of the above aspects the heterocyclic compound is selected from:

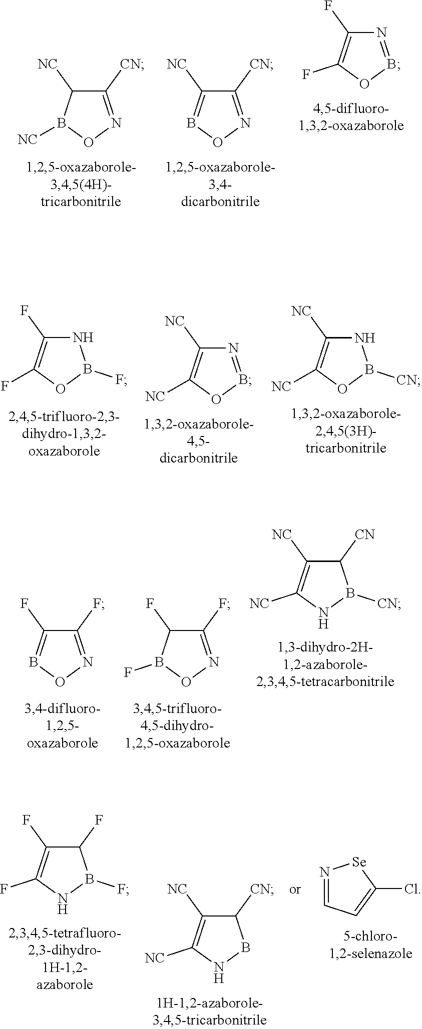

In any or all of the above aspects, two of J, Q, T, X, or Y are N and/or N(R′), wherein R″ is hydrogen; and the remaining J, Q, T, X, or Y groups are (i) B; (ii) BR^{c}; (iii) O; (iv) Se; and/or (v) CR^{c}, wherein R^{c} is hydrogen, cyano, or halo.

In any or all of the above aspects, the heterocyclic compound is selected from:

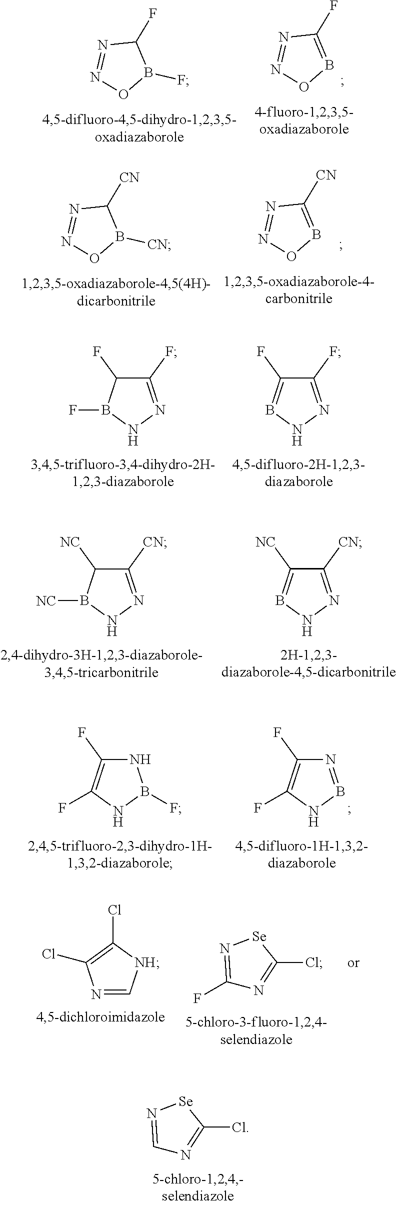

In any or all of the above aspects two of J, Q, T, X, or Y are independently (i) S and (ii) B or BR^{c}.

In any or all of the above aspects the heterocyclic compound is selected from:

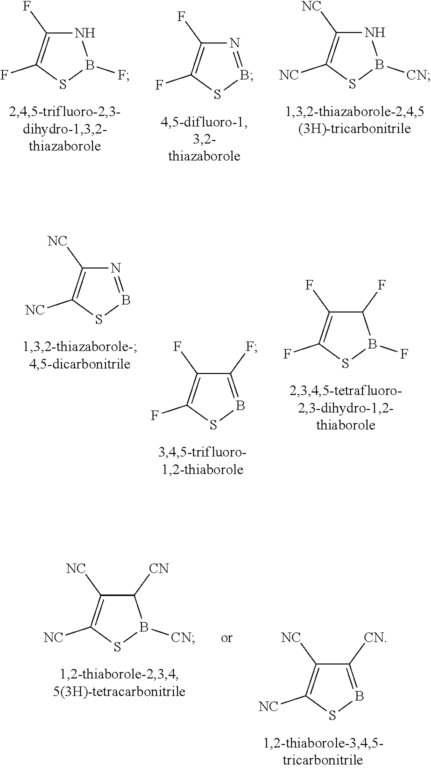

In any or all of the above aspects, one or more of J, Q, T, X, or Y is nitrogen; one or more of J, Q, T, X, or Y is N(R″) wherein the R″ group is hydrogen, aliphatic, or cycloaliphatic; and at least two of J, Q, T, X, or Y individually is CR^{c}.

In any or all of the above aspects, the R^{c} groups of the two CR^{c} groups, together with the carbon atoms to which they are bound, form an aryl or heteroaryl ring system.

In any or all of the above aspects, the aryl or heteroaryl ring system further comprises a halo substituent.

In any or all of the above aspects, the heterocyclic compound is selected from:

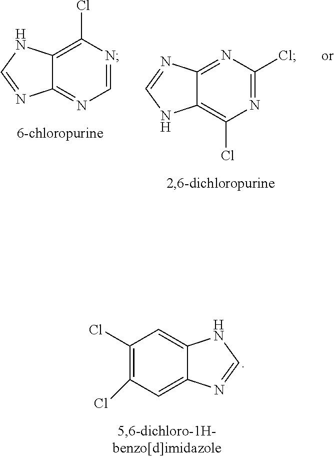

In any or all of the above aspects, where m is 1, and the heterocyclic compound has a structure according to Formula III

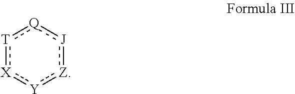

In any or all of the above aspects, at least one of J, Q, T, X, Y, or Z is N, N(R″), or N⁺[R″] and at least one of J, Q, T, X, Y, or Z is B or BR^{c}, wherein the R^{c} group is halo or cyano.

In any or all of the above aspects, the halo is F or Cl.

In any or all of the above aspects, the heterocyclic compound is selected from:

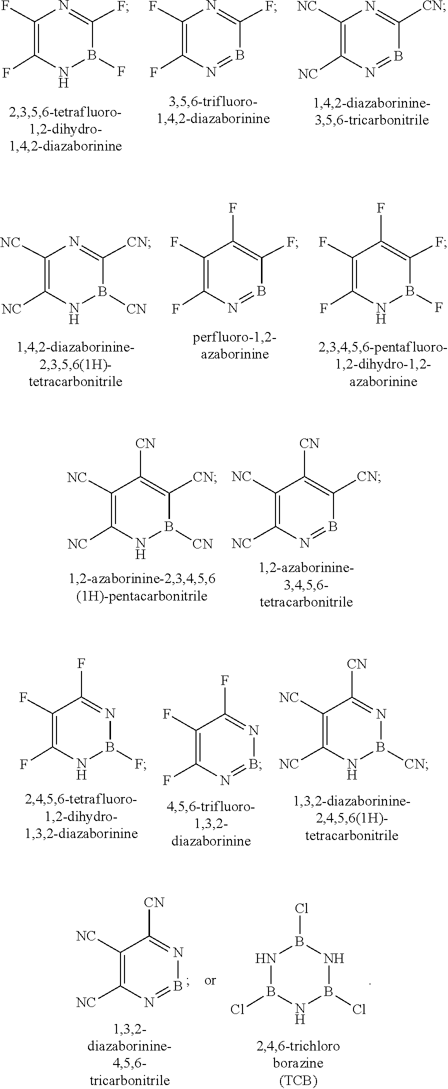

In any or all of the above aspects, at least one of J, Q, T, X, Y, or Z is N or N⁺[R″] and at least two of J, Q, T, X, Y, or Z are CR^{c}, wherein each R^{c} group independently is selected from halo, heteroaryl, carbonyl, nitro, nitrile, carboxyl, isocyanate, oxime, amino, alkoxy, or aralkyl.

In any or all of the above aspects, at least two of J, Q, T, X, Y, or Z are CR^{c}; and wherein at least one R^{c} group is halo.

In any or all of the above aspects, the halo is Cl or Br.

In any or all of the above aspects, the heterocyclic compound is selected from:

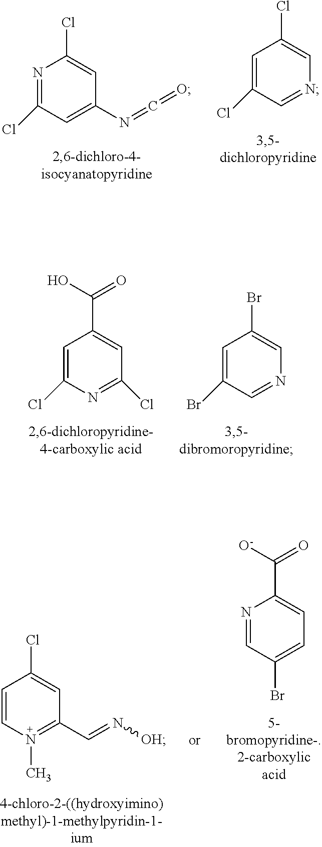

In any or all of the above aspects, at least two of J, Q, T, X, Y, or Z are N.

In any or all of the above aspects, at least one of J, Q, T, X, Y, or Z is CR^{c}; and wherein R^{c} is halo.

In any or all of the above aspects, at least two of J, Q, T, X, Y, or Z are CR^{c}; and wherein the two R^{c} groups are halo.

In any or all of the above aspects, J, Q, T, X, Y, or Z are selected such that two of J, Q, T, X, Y, or Z are nitrogen separated by one CR^{c} group.

In any or all of the above aspects, the R^{c} group is hydrogen.

In any or all of the above aspects, the heterocyclic compound is selected from:

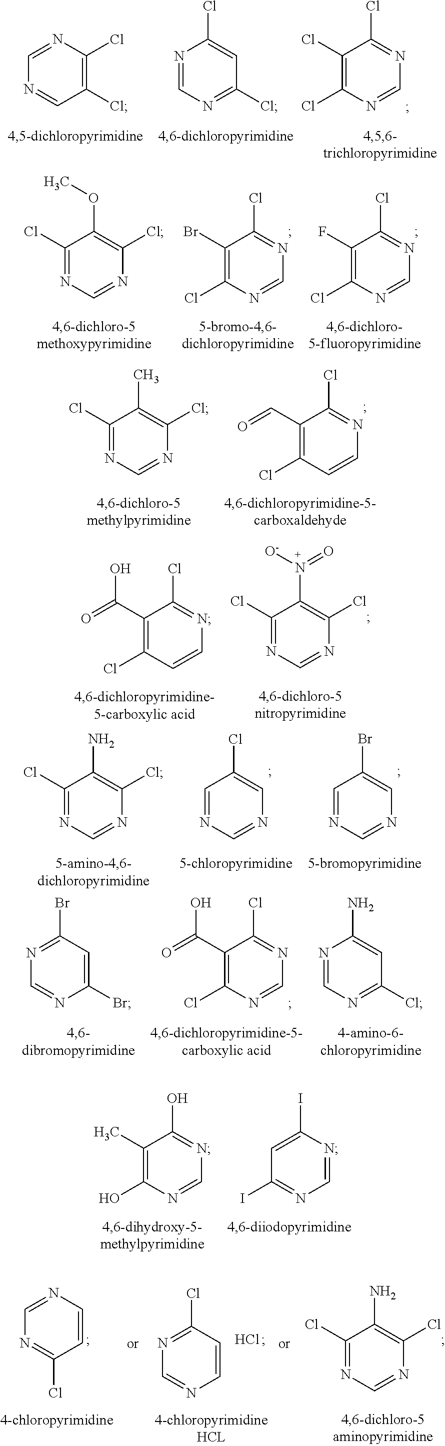

In any or all of the above aspects, the R^{c} group is other than hydrogen.

In any or all of the above aspects, the heterocyclic compound is selected from:

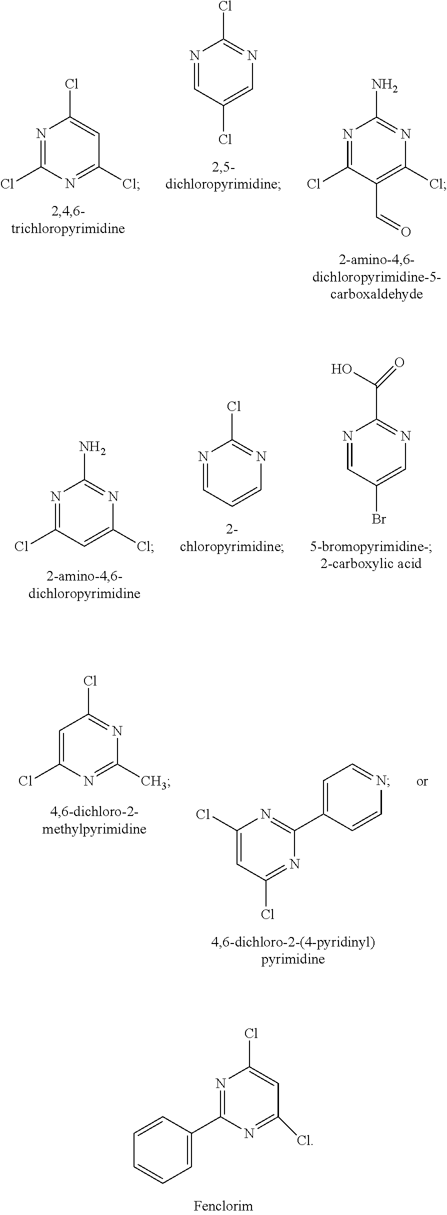

In any or all of the above aspects, J, O, T, X, Y, or Z are selected such that two of J, O, T, X, Y, or Z are nitrogen separated by two CR^{c} groups.

In any or all of the above aspects, the heterocyclic compound is selected from:

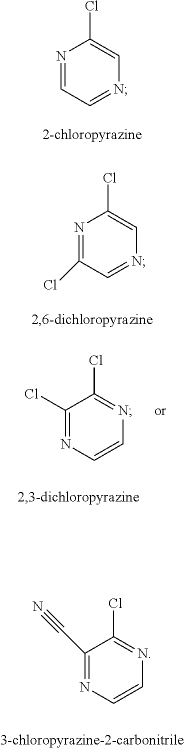 [SMILES: Clc1cnccn1 ; Clc1cncc(Cl)n1 ; Clc1nccnc1Cl ; N#Cc1nccnc1Cl]

In any or all of the above aspects, the composition further comprises a buffer.

In any or all of the above aspects, the buffer is selected from bis(2-hydroxyethyl)iminotris(hydroxymethyl)methane, tris-glycine, tris-acetate, or any combination thereof.

In any or all of the above aspects, the composition further comprises a polyacrylamide compound.

In any or all of the above aspects, the polyacrylamide is a polymer network derived from bisacrylamide and acrylamide.

In any or all of the above aspects, the composition further comprises an initiator, promoter, or combination thereof.

In any or all of the above aspects, the initiator is ammonium persulfate and the promoter is N,N,N′,N′-tetramethylethylenediamine.

In any or all of the above aspects, the concentration of the heterocyclic compound in the composition ranges from 0.001% to 5% by volume.

Also disclosed herein is a method for making a composition for detecting one or more proteins in a sample, comprising: adding the composition disclosed herein to a container; adding a monomer and a crosslinker to the container; adding an initiator, promoter, or combination thereof, to the container to promote forming a polymer network derived from the monomer and the crosslinker; and adding a buffer to the container.

In some aspects of the present disclosure, the monomer is acrylamide and the crosslinker is bisacrylamide (N,N′-methylenediacrylamide).

In any or all of the above aspects, the acrylamide and bisacrylamide are used in amounts providing a ratio of 20:1 to 40:1 (acrylamide:bisacrylamide).

In any or all of the above aspects, the initiator is ammonium persulfate.

In any or all of the above aspects, the promoter is N,N,N′,N′-tetramethylethylenediamine.

In any or all of the above aspects, the buffer is selected from bis(2-hydroxyethyl)iminotris(hydroxymethyl)methane, tris-glycine, tris-acetate, or any combination thereof.

Also disclosed herein is a method for detecting one or more biomolecules in a sample, comprising adding the sample to a container comprising (i) the composition disclosed herein, (ii) a crosslinked polymer network, and (iii) a buffer; exposing the container to an energy source; and detecting the presence of an amino acid conjugate formed upon association of the heterocyclic compound with the aromatic amino acid residue of the biomolecule.

In any or all of the above aspects, the heterocyclic compound is covalently bound to the aromatic amino acid residue of the biomolecule.

In any or all of the above aspects, a fluorescent signal is produced after exposing the container to the energy source for a time period ranging from 1 millisecond to 600,000 milliseconds.

In any or all of the above aspects, the energy source is a light source capable of producing visible light or UV light.

Also disclosed herein is a composition comprising a buffer; a polyacrylamide derived from acrylamide and bisacrylamide; a biomolecule comprising an aromatic amino acid residue; and an associating means for producing an amino acid conjugate upon associating with the aromatic amino acid residue.

Also disclosed herein is a kit for detecting one or more biomolecules, comprising a container comprising the composition disclosed herein; and instructions for using the composition.

In some aspects the kit further comprises polyacrylamide.

In any or all of the above aspects, the polyacrylamide is derived from acrylamide and bisacrylamide.

In any or all of the above aspects, the kit further comprises a buffer.

In any or all of the above aspects, the buffer is bis(2-hydroxyethyl) iminotris(hydroxymethyl)methane, tris-glycine, tris-acetate, or any combination thereof.

### VIII. Examples

Spectroscopic Materials and Methods: Untreated lysozyme samples (control) and treated lysozyme with a heterocyclic compound disclosed herein were reduced and alkylated; acetone precipitated; and digested with Trypsin/Lys-C for 1 hour at (1:20 ratio). The samples were then acidified and dried on a speed vacuum and reconstituted with 0.1% formic acid. Next, the samples were run on QEPlus instrument and searched using Sequest HT search engine with the modifications on PD 3.0 software. PD data was imported and ran the samples with a targeted PRM approach on QEHF. The data was analyzed on Skyline.

Workflow 1: To quantitate macromolecules (e.g., protein) in solution or other media (e.g., gels), (1) a heterocyclic compound disclosed herein was added to the lysozyme containing solution in a tube, cuvette, microplate, etc. and activated/exposed to UV light; (2) imaged and/or scanned for fluorescence; (3) compared to a reference standard. The labeled lysozyme can be prepared for PAGE, electrophoresed, and imaged without further UV activation.

Workflow 2: The heterocyclic compound was used as a stain for PAGE (Polyacrylamide Gel Electrophoresis) by (1) adding the heterocyclic compound to a gel casting solution and casting the gel; (2) the samples were electrophoresed; (3) activated/exposed to UV light; and (4) imaged to visualize fluorescence.

Workflow 3: To assess the efficiency of the gel to membrane transfer, Workflow 2 was first performed. Next the UV-activated gel was transferred (using either a wet, semi-dry, or dry transfer method) to a PVDF membrane. The membrane was then imaged and/or the post-transfer gel was visualized by fluorescence and compared to results obtained in Workflow 2.

Workflow 4: For normalizing immunodetection results from blots (membranes) to total protein, Workflow 2 was first performed. Using a wet, semi-dry, or dry transfer method the UV-activated gel was transferred to a PVDF membrane. The PVDF membrane was then imaged or visualized by fluorescence; with immunodetection being performed according to standard western protocols. Finally, the immunodetected membrane was imaged to compare the antibody-dependent (immunodetected) signal to the total protein fluorescent signal.

### Example 1

In this example, the fluorescence emission spectra of (i) trichloroethanol (TCE) with N-acetyl tryptophan in methanol; and (ii) β-butyrolactone with N-acetyl tryptophan in methanol were obtained and then compared to the spectra of 4,6-dichloropyrimidine (4,6-DiPy) with N-acetyl tryptophan in methanol.

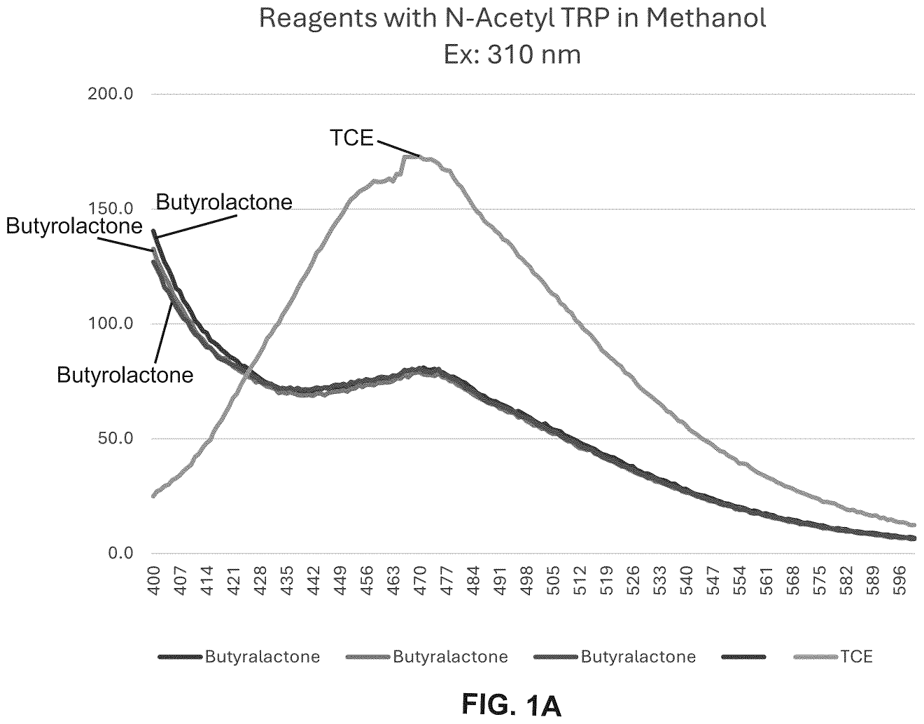

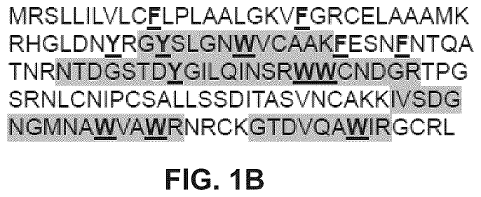

FIG. 1A is the fluorescence emission spectra of 4,6-dichloropyrimidine compared to that of TCE and β-butyrolactone. As can be seen, the 4,6-dichloropyrimidine exhibited a different peak wavelength in comparison to TCE and β-butyrolactone; thus, 4,6-dichloropyrimidine exhibited a different signature, which further demonstrated the formation of different products for TCE, 4,6-dichloropyrimidine, and β-butyrolactone.

### Example 2

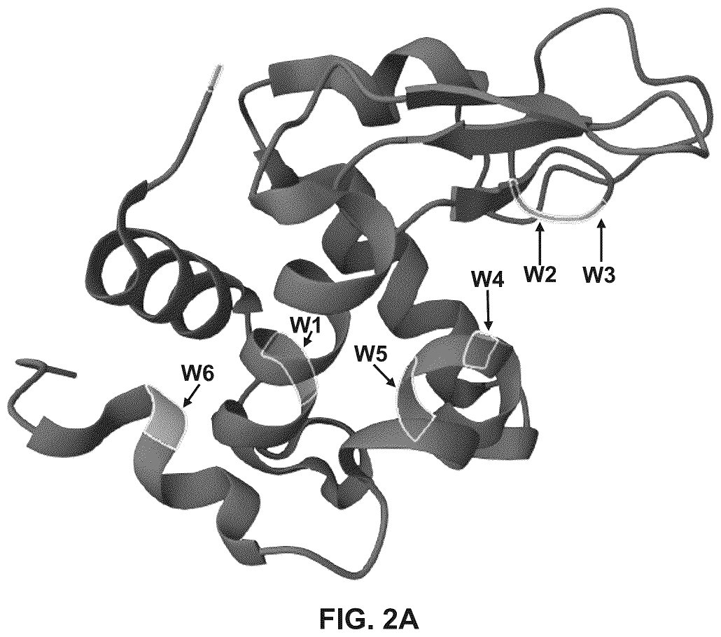

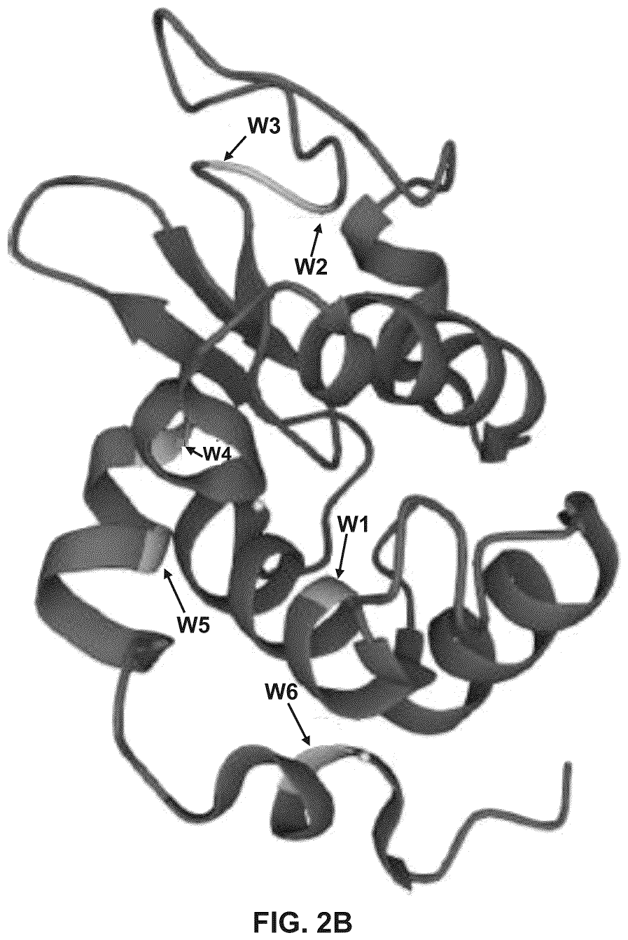

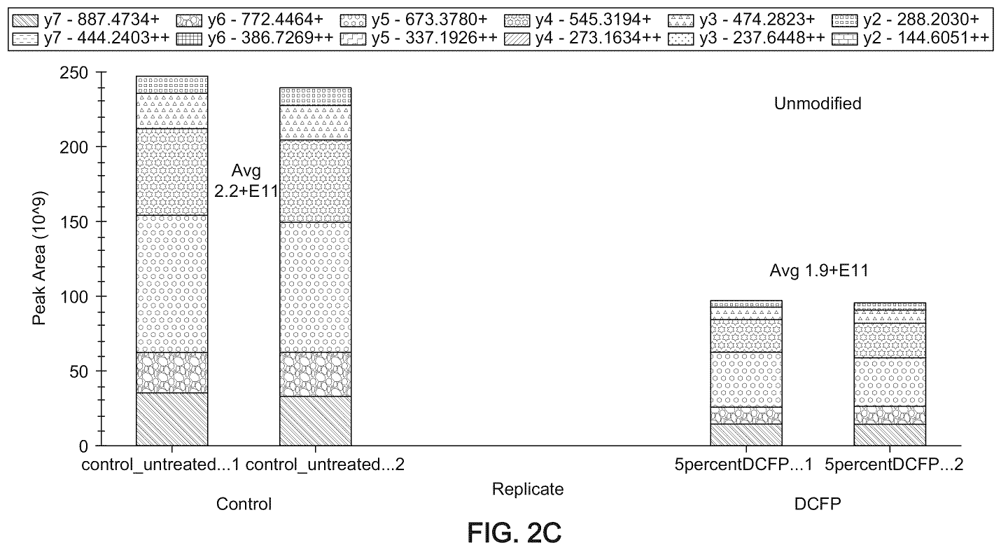

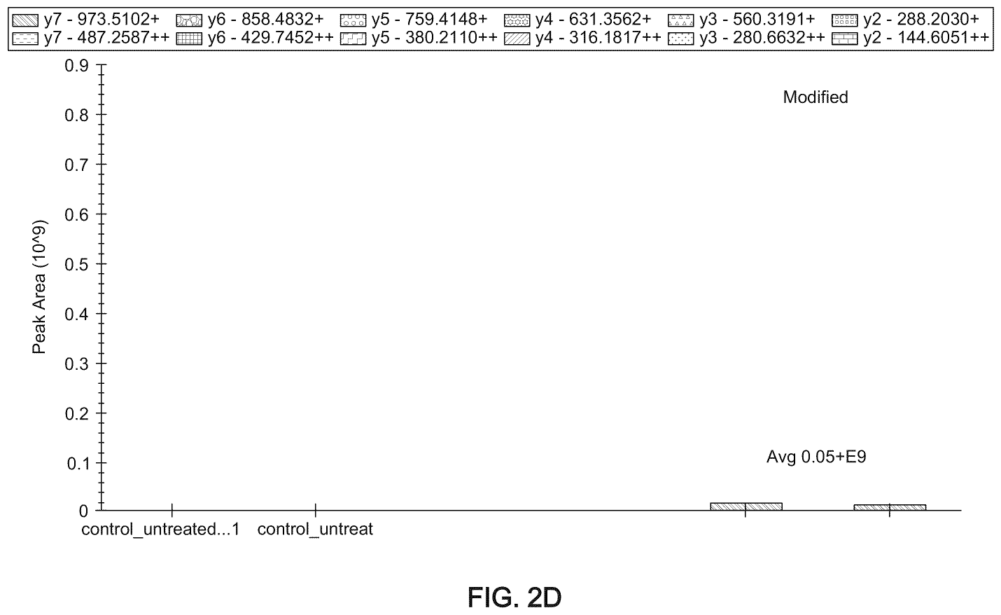

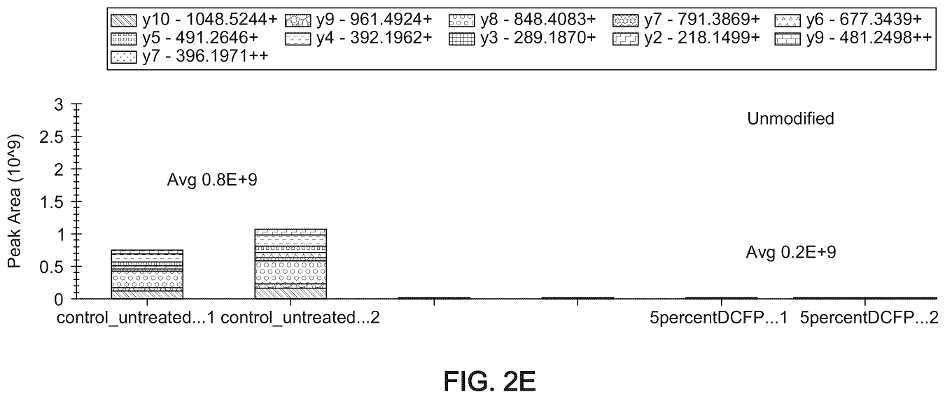

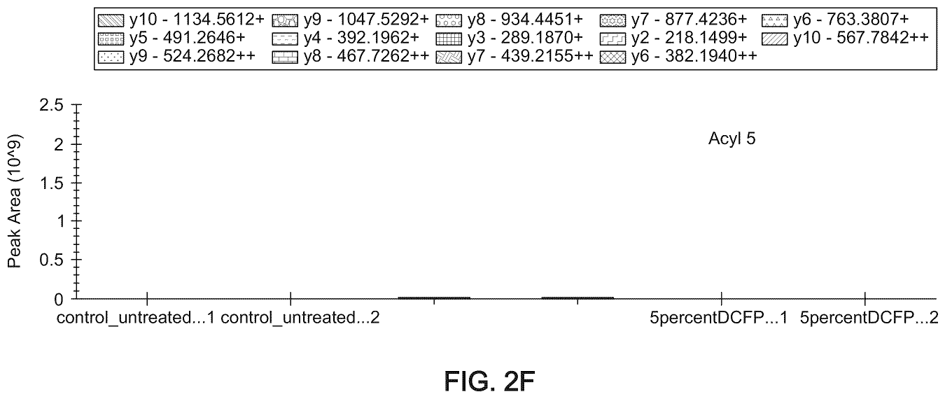

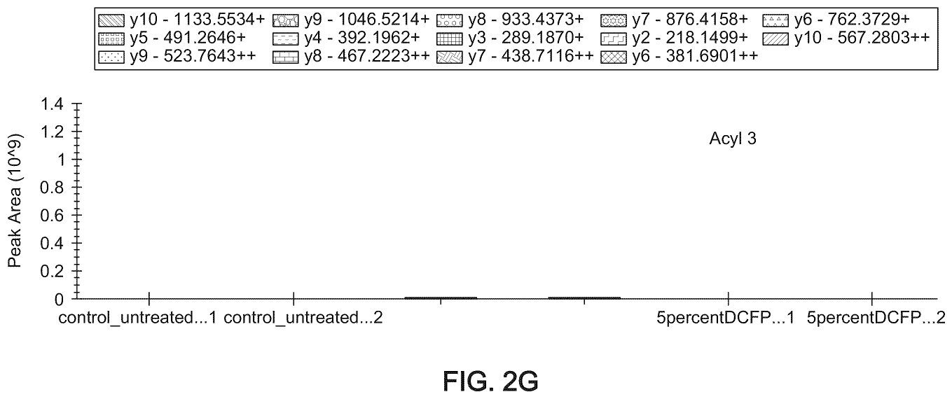

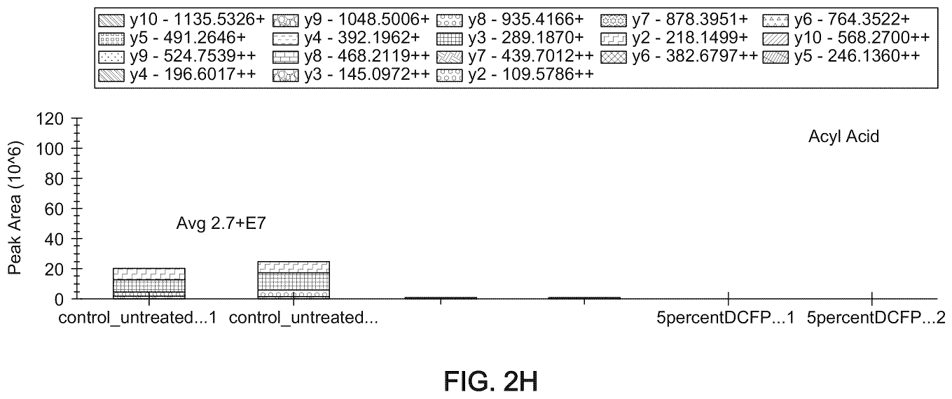

In this example, a sample comprising 4,6-dichloro-5-fluoropyrimidine (DCFP), and a folded native protein, chicken lysozyme (P00698, 16.2 kDa, 151 amino acids) (SEQ ID NO: 1), which is illustrated in FIGS. 2A-2B, was investigated to analyze modifications to aromatic amino acid residues (SEQ ID: NO 2) (i.e., tryptophan (W)). The MS data is shown in Table 1, demonstrating modifications on W1, W2, W3, and W6.

FIG. 2C is a bar graph showing the peak area intensity of fragment having SEQ ID NO: 3 (unmodified) comparing the abundance in the control (unmodified) sample versus the abundance in the DCFP sample (modified). The bar graph in FIG. 2C demonstrates that the unmodified peptide abundance (average 2.2E11) is higher in control samples compared to the DCFP treated samples (average 1.9E11).

FIG. 2D is a bar graph showing the peak area intensity of fragment having SEQ ID NO: 4 (modified) comparing the abundance in the control sample (unmodified) versus the abundance in the DCFP sample (modified). The bar graph in FIG. 2D demonstrates that the modified peptide abundance (average 0.05E9) is higher in DCFP treated samples.

FIG. 2E is a bar graph showing the peak area intensity of fragment having SEQ ID NO: 5, which compares the abundance in the control (unmodified) sample and the abundance in the DCFP-modified sample. The bar graph of FIG. 2E demonstrates that the unmodified peptide abundance (average 0.8E9) is higher in control samples.

FIG. 2F is a bar graph showing the peak area intensity of fragment having SEQ ID NO: 6, which compares the abundance in the control (unmodified) sample and the abundance in the DCFP-modified sample. The bar graph of FIG. 2F demonstrates that the modified peptide abundance was undetected, similar to the unmodified peptide in DCFP treated samples.

FIG. 2G is a bar graph showing the peak area intensity of fragment SEQ ID NO: 7, which compares the abundance in the control (unmodified) sample and the abundance in the DCFP-modified sample. The bar graph of FIG. 2G demonstrates that the modified peptide abundance was undetected, similar to the unmodified peptide in DCFP treated samples.

FIG. 2H is a bar graph showing the peak area intensity of fragment SEQ ID NO: 8, which compares the abundance in the control (unmodified) sample and the abundance in the DCFP-modified sample. The bar graph of FIG. 2H demonstrates that the unmodified peptide abundance (average 2.7E7) was higher in DCFP treated samples.

FIG. 2I is a bar graph showing the peak area intensity of fragment SEQ ID NO: 10, which compares the abundance in the control (unmodified) sample having SEQ ID: NO 9 and the abundance in the DCFP-modified sample. FIG. 2I demonstrates that the modified peptide abundance (average 0.1E6) was higher in DCFP treated samples.

TABLE 1

| | Control - Untreated Chicken Lysozyme | | Replicate - DCFP treated Chicken Lysozyme | |
|---|---|---|---|---|
| | (SEQ ID NO: 1) | | (SEQ ID NO: 2) | |
| | 1 | 2 | 1 | 2 |
| W1 | Acyl3 | Acyl3 | Acyl 5 | |
| | (85.0289) | (85.0289) | (86.0453) | |
| | Acyl4 | Acyl4 | Chlorofluoroamine | Chlorofluoroamine |
| | (69.0304) | (69.0304) | (121.9808) | (121.9808) |
| | | Acyl 5 | | |
| | | (86.0453) | | |
| | Acyl 6 | Acyl 6 | | |
| | (87.0453) | (87.0453) | | |
| | Acyl acid | Chlorohydroxyimine | | |
| | (87.0082) | (86.0242) | | |
| | Extended carbonyl | Chloroaromatic | | |
| | (71.0133) | (112.9906) | | |
| W2, | | | Acyl 5 | Chlorofluoroamine |
| W3 | | | (86.0453) | (121.9808) |
| W4, | | | | |
| W5 | | | | |
| W6 | Chloroaromatic | | Acyl 5 | Acyl 5 |
| | (112.9906) | | (86.0453) | (86.0453) |

### Example 3

In this example, a hand-cast and electrophoresed Bis-Tris gel comprising 4,6-dichloropyrimidine (0.1%) was imaged with no UV-activation period and then imaged after a 5-minute UV-activation period; these images were compared to images acquired from a hand-cast and electrophoresed Bis-Tris gel comprising TCE (0.1%) that were first imaged with no UV-activation period and then imaged after a 5-minute UV activation period, wherein the gels were loaded and electrophoresed with four different cellular lysate samples (namely, HeLa, E. coli, HEK293, and rat liver).

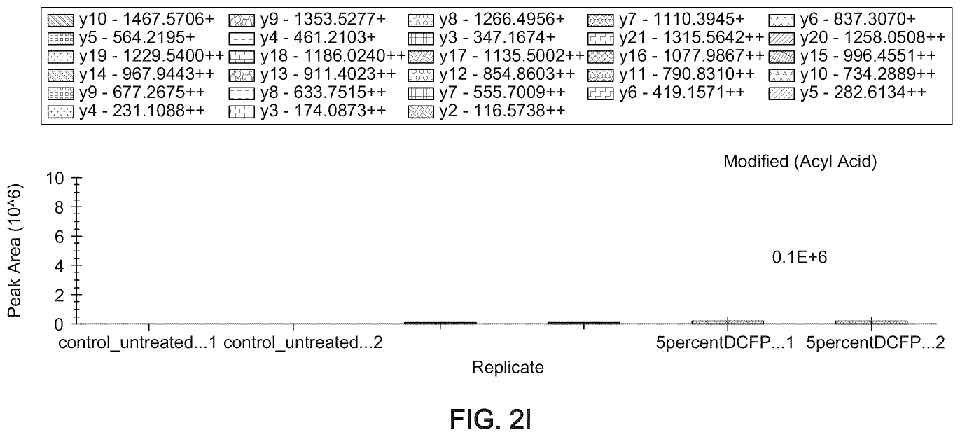

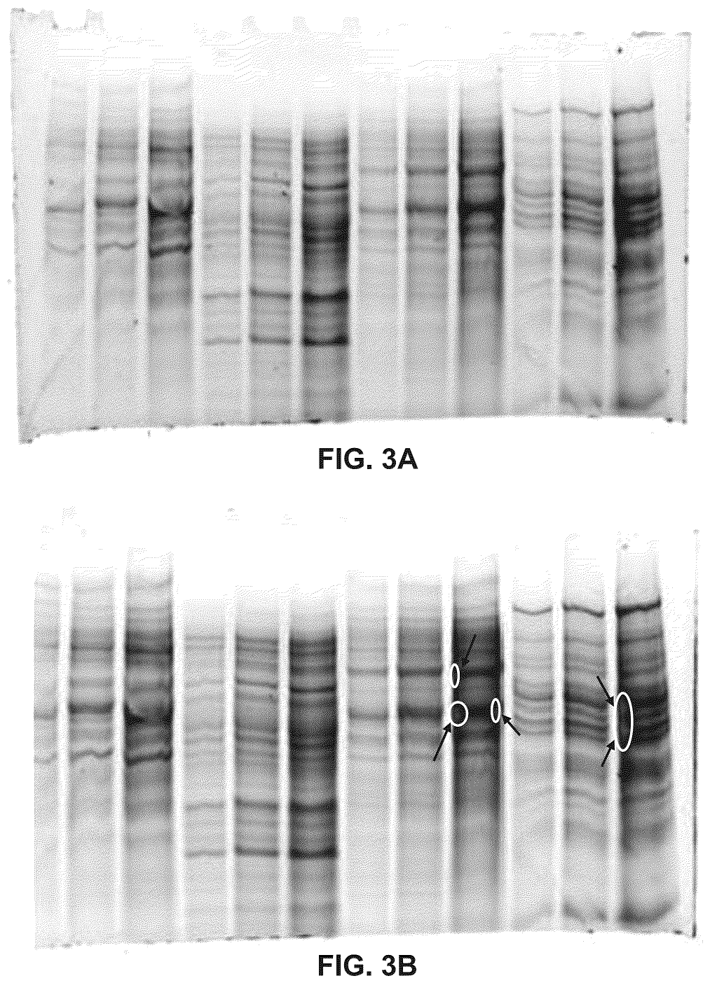

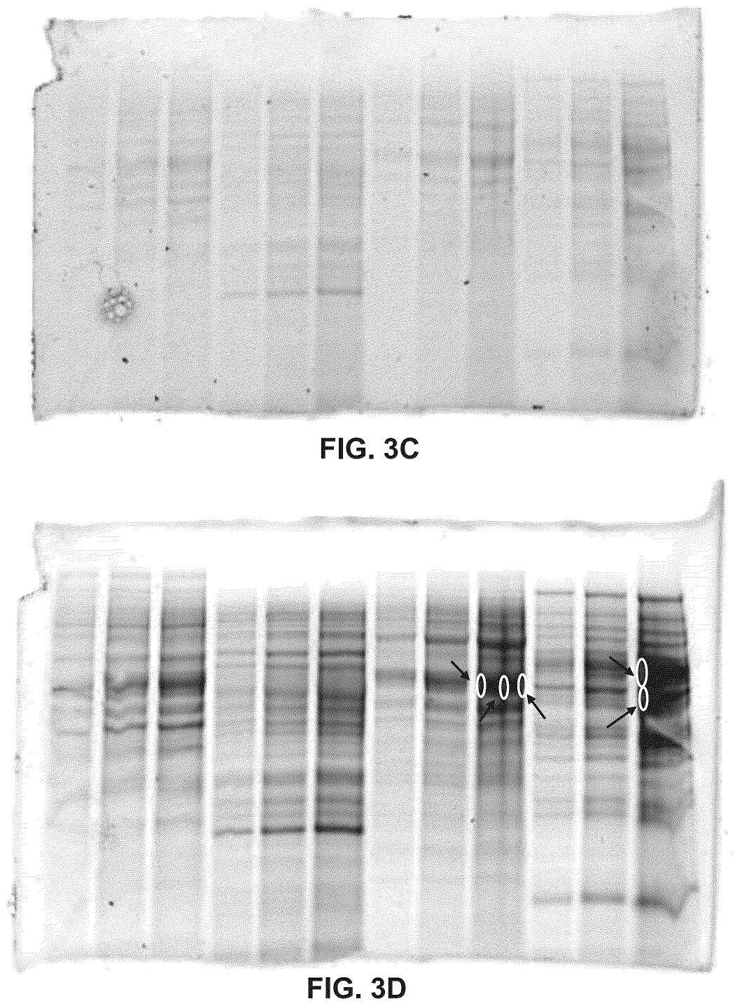

FIG. 3A is a digital image of an electrophoresed Bis-Tris gel (with no UV activation period) comprising 4,6-dichloropyrimidine (0.1%) and acquired using a 5-second manual exposure. FIG. 3B is a digital image of the electrophoresed Bis-Tris gel shown in FIG. 3A (imaged following a 5-minute UV activation period) comprising 4,6-dichloropyrimidine (0.1%) and imaged using an auto (optimal) exposure time of 2.168 seconds. FIG. 3C is a digital image of an electrophoresed Bis-Tris gel (with no UV activation period) comprising TCE (0.1%) and acquired using a manual exposure time of 5 seconds. FIG. 3D is a digital image of the electrophoresed Bis-Tris gel shown in FIG. 3C (following a 5-minute UV activation period) comprising TCE (0.1%) and imaged using an auto (optimal) exposure time of 6.275 seconds.

As shown by FIGS. 3A-3D, a greater signal intensity was demonstrated with the Bis-Tris gel comprising 4,6-dichloropyrimidine (0.1%) compared to the gel comprising TCE (0.1%) when imaged with no UV activation period and using a manual exposure time of 5 seconds thereby demonstrating a greater signal intensity and more robust reactivity of 4,6-dichloropyrimidine relative to TCE at the same in-gel concentration. Moreover, the auto (optimal) exposure time for the UV-activated 4,6-dichloropyrimidine (0.1%) gel was threefold less than that of the TCE (0.1%) gel; thereby indicating a greater capacity to obtain stronger signal intensities with gels comprising 4,6-dichloropyrimidine (0.1%) over gels comprising TCE (0.1%).

### Example 4

In this example, a hand-cast and electrophoresed Bis-Tris gel comprising 4,6-dichloropyrimidine (0.02%) and imaged with no UV-activation period and followed by a 45-second UV-activation period was compared to a hand-cast and electrophoresed Bis-Tris gel comprising TCE (0.02%) with no UV-activation period and a 45-second UV-activation period with both gels being loaded and electrophoresed with four different cellular lysate samples (namely, HeLa, E. coli, HEK293, and rat liver). Following electrophoresis and imaging, gels were transferred to PVDF membranes, which were then imaged using auto (optimal) exposures.

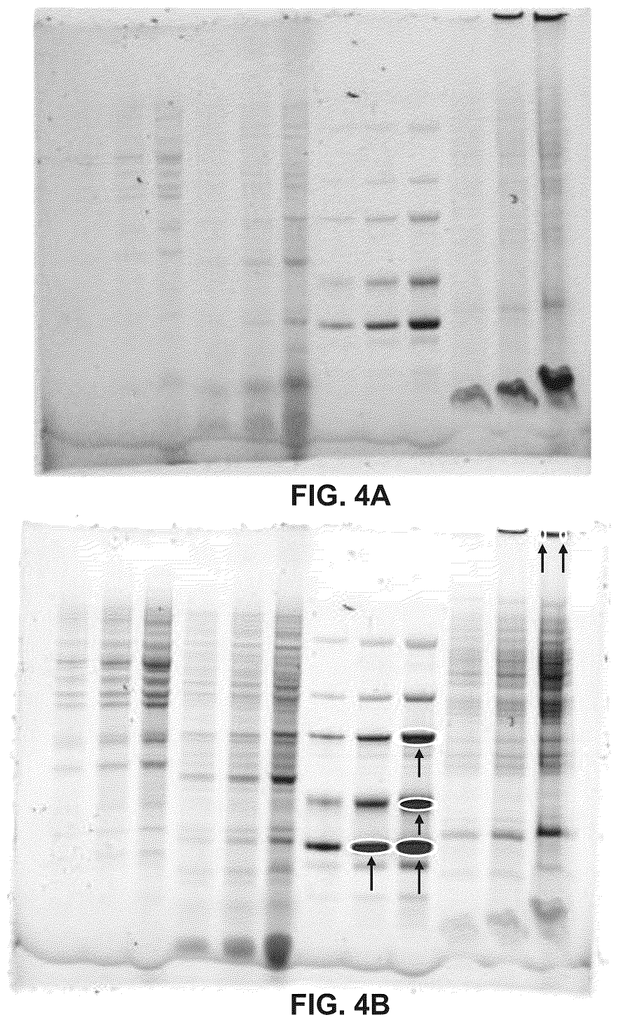

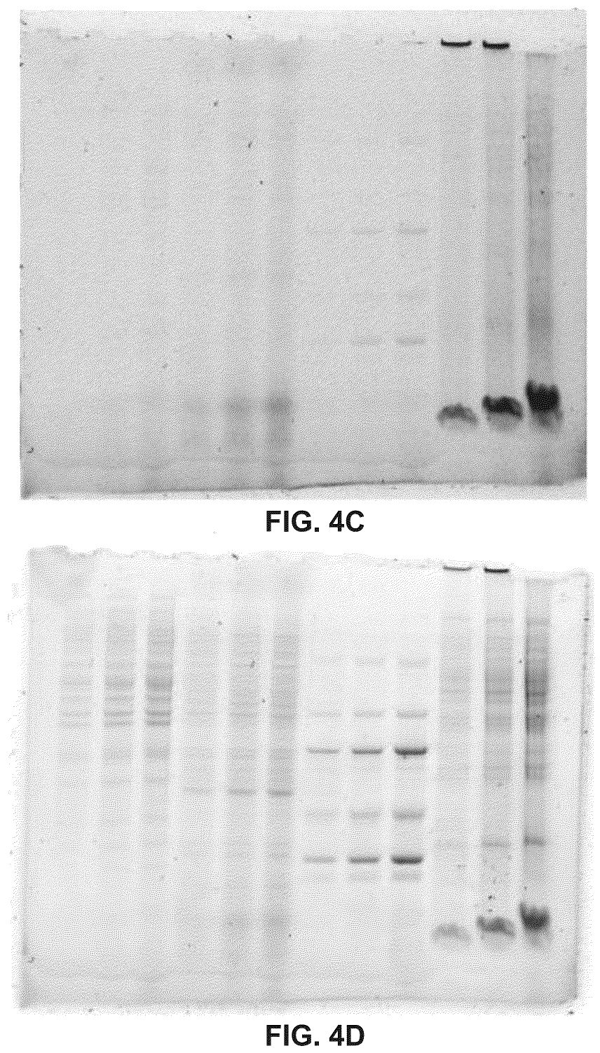

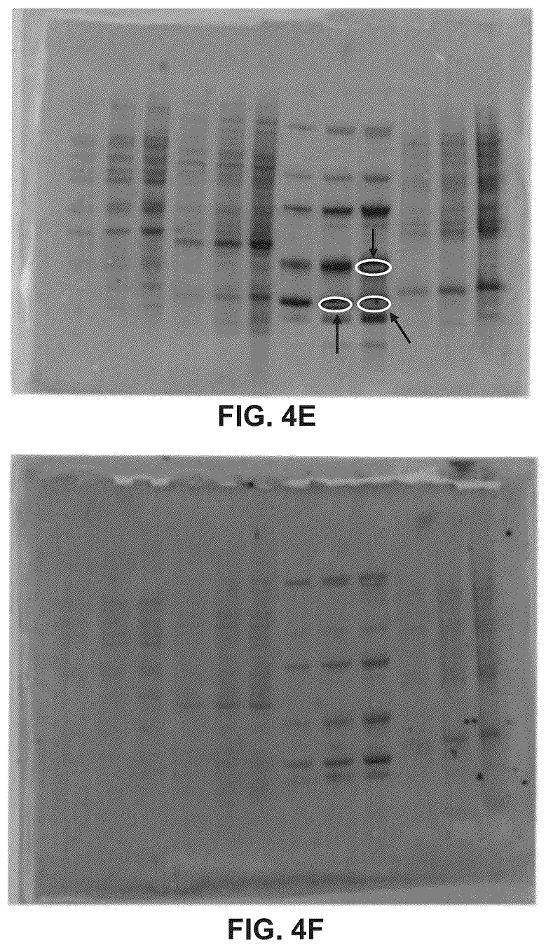

FIG. 4A is a digital image of an electrophoresed Bis-Tris gel with no UV activation period comprising 4,5,6-trichloropyrimidine (0.02%) and imaged with a manual exposure time of 0.5 seconds. FIG. 4B is a digital image of an electrophoresed Bis-Tris gel with a 45-second UV activation period comprising 4,5,6-trichloropyrimidine (0.02%) and imaged after the 45-second UV activation period with an auto optimal exposure time of 13.319 seconds. FIG. 4C is a digital image of an electrophoresed Bis-Tris gel with no UV activation period comprising TCE (0.02%) and imaged with a manual exposure time of 0.5 second. FIG. 4D is a digital image of an electrophoresed Bis-Tris gel with a 45-second UV activation period comprising TCE (0.02%) and imaged after the 45-second UV activation period with an auto optimal exposure time of 13.319 seconds. FIG. 4E is a digital image of a membrane comprising the electrophoresed gel of FIG. 4B and imaged with an auto optimal exposure time of 3.543 seconds. FIG. 4F is a digital image of a membrane comprising the electrophoresed gel of FIG. 4D and imaged with an auto optimal exposure time of 3.543 seconds.

In view of FIGS. 4A-4D, gels comprising 4,5,6-trichloropyrimidine (0.02%) exhibited stronger signal intensity and sensitivity than the gels comprising TCE (0.02%). Additionally, as can be seen in FIGS. 4E-4F, the PVDF membrane from the gel comprising 4,5,6-trichloropyrimidine (0.02%) demonstrated greater signal intensity and sensitivity than the PVDF membrane from the gel comprising TCE (0.02%).

### Example 5

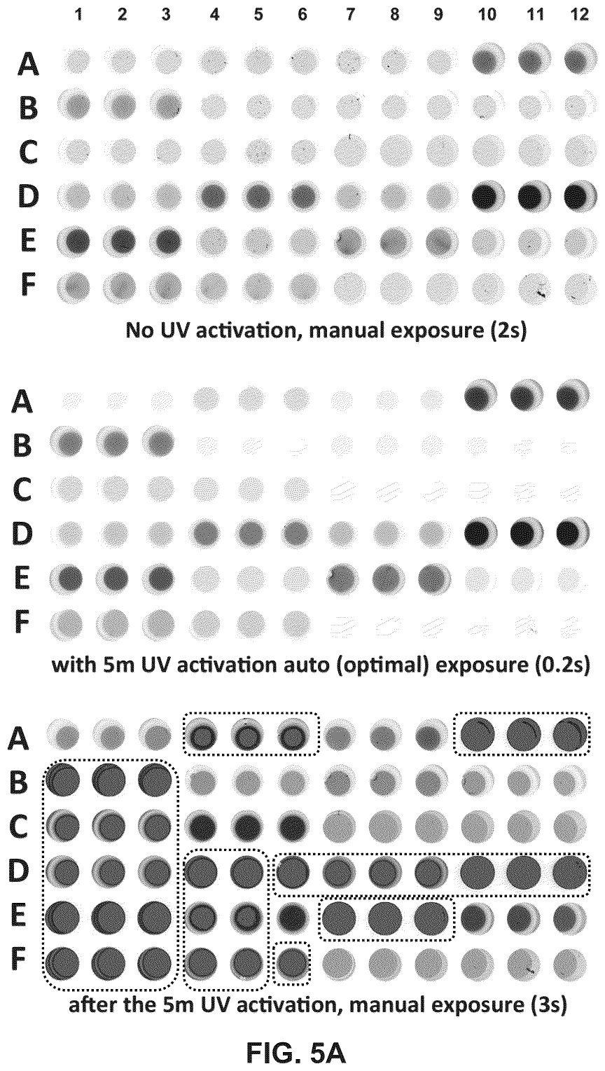

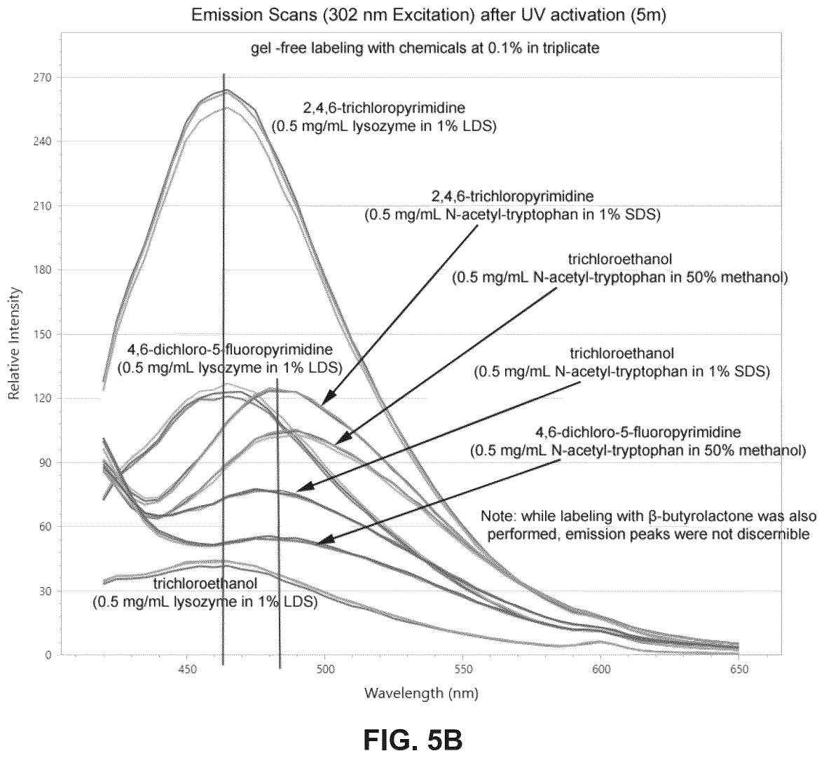

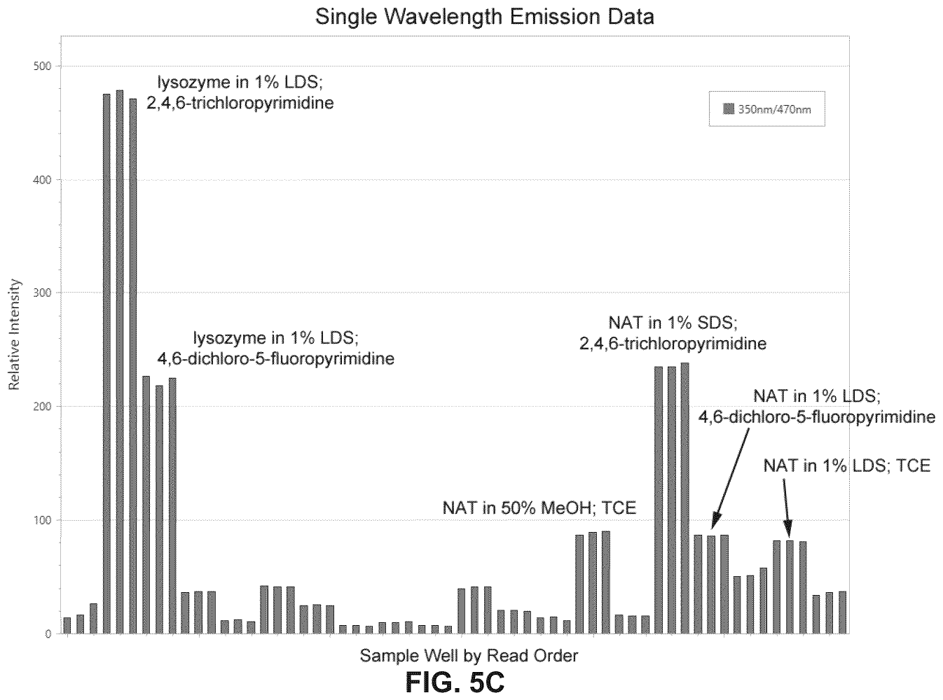

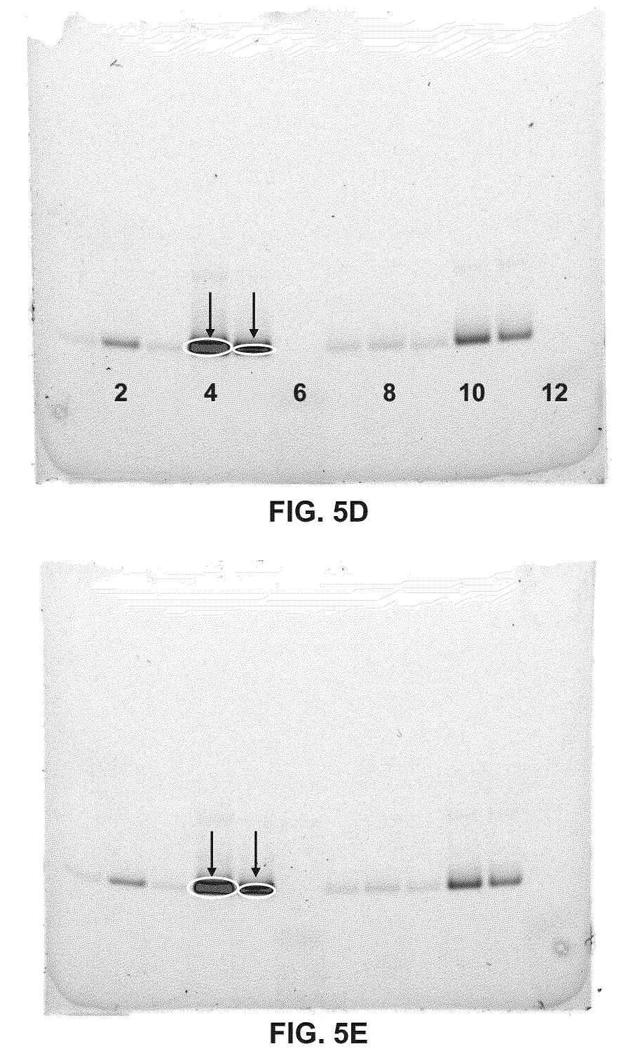

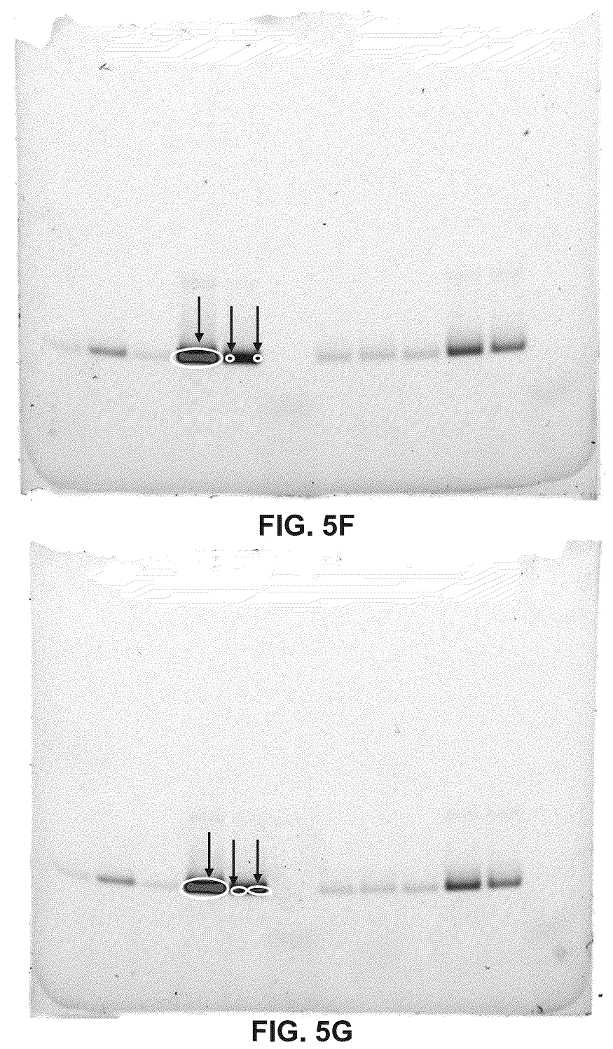

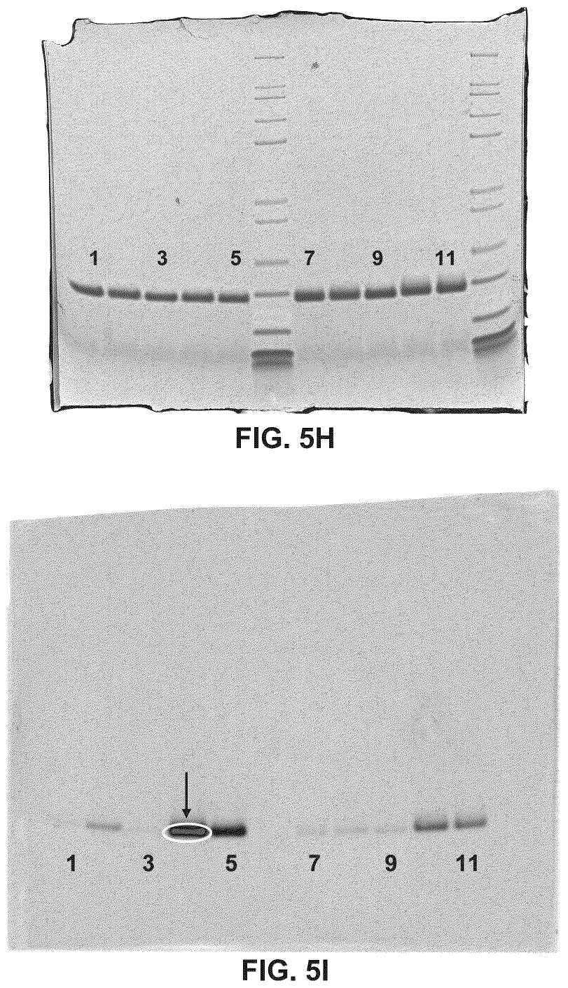

In this example, a 96-well plate was configured according to Table 2 and comprised unlabeled lysozyme in 1% LDS, unlabeled N-acetyl-tryptophan (NAT), lysozyme labeled with 4,6 dichloro-5-fluoropyrimidine, lysozyme labeled with 2,4,6-trichloropyrimidine, lysozyme labeled with β-butyrolactone, NAT labeled with 4,6 dichloro-5-fluoropyrimidine, NAT labeled with 2,4,6-trichloropyrimidine, and NAT labeled with β-butyrolactone with (i) no UV activation period and imaged with a manual exposure time of 2 seconds (top well-plate image of FIG. 5A), (ii) a 5-minute UV activation period and imaged using an auto (optimal) exposure time of 0.2 seconds (middle well-plate image of FIG. 5A) and (iii) no additional 5-minute UV activation period but imaged using a manual exposure time of 3 seconds (bottom well-plate image of FIG. 5A).

TABLE 2

| | 1 | 2 | 3 | 4 | 5 | 6 | 7 | 8 | 9 | 10 | 11 | 12 |
|---|---|---|---|---|---|---|---|---|---|---|---|---|
| A | Lysozyme in 1% | | | Lysozyme in 1% | | | Lysozyme in 1% LDS, | | | Lysozyme in 1% LDS, | | |
| | LDS (unlabeled) | | | LDS, TCE | | | β-butyrolactone | | | 2,4,6-trichloropyrimidine | | |
| B | Lysozyme in 1% | | | Lysozyme in 50% | | | Lysozyme in 50% | | | Lysozyme in 50% MeOH | | |
| | LDS, | | | MeOH (unlabeled) | | | MeOH, TCE | | | | | |
| | 4,6 dichloro-5- | | | | | | | | | | | |
| | fluoropyrimidine | | | | | | | | | | | |
| C | Lysozyme in 50% | | | Lysozyme in 50% | | | empty | empty | empty | empty | empty | empty |
| | MeOH, 2,4,6- | | | MeOH, 4,6 | | | | | | | | |
| | trichloropyrimidine | | | dichloro-5- | | | | | | | | |
| | | | | fluoropyrimidine | | | | | | | | |
| D | NAT in 1% SDS | | | NAT in 1% SDS, | | | NAT in 1% SDS, | | | NAT in 1% SDS, | | |
| | (unlabeled) | | | TCE | | | β-butyrolactone | | | 2,4,6-trichloropyrimidine | | |
| E | NAT in 1% SDS, | | | NAT in 50% | | | NAT in 50% MeOH, TCE | | | NAT in 50% MeOH | | |
| | 4,6 dichloro-5- | | | MeOH, | | | | | | | | |
| | fluoropyrimidine | | | (unlabeled) | | | | | | | | |
| F | NAT in 50% MeOH, | | | NAT in 50% | | | empty | empty | empty | empty | empty | empty |
| | 2,4,6- | | | MeOH, | | | | | | | | |
| | trichloropyrimidine | | | 4,6 dichloro-5- | | | | | | | | |
| | | | | fluoropyrimidine | | | | | | | | |

FIG. 5A is an illustration of the qualitative results of plate labeling followed by imaging (i) with no UV activation, manual exposure (2 seconds); (ii) a 5-minute UV activation period, auto (optimal) exposure (0.2 seconds); and (iii) no additional 5-minute UV activation, manual exposure (3 seconds). As shown qualitatively by FIG. 5A, a strong signal intensity was demonstrated after using plate labeling with no UV activation period and imaging with a manual exposure (2 seconds) for samples comprising 2,4,6-trichloropyrimidine (wells D10-D12) or 4,6 dichloro-5-fluoropyrimidine (wells E1-E3). Less signal intensity was observed with the samples comprising TCE (wells D4-D6 and E7-E9). For samples comprising 2,4,6-trichloropyrimidine (wells C1-C3) or 4,6 dichloro-5-fluoropyrimidine (wells A10-A12), plate labeling with a 5-minute UV activation period and imaging with auto (optimal) exposure (0.2 seconds) demonstrated increased signal intensity compared to imaging the plate with no UV activation period and imaged with a manual exposure (2 seconds). Strong signal intensities were observed for samples comprising 2,4,6-trichloropyrimidine, 4,6 dichloro-5-fluoropyrimidine using plate labeling with a 5-minute UV activation period and imaged with a manual exposure (3 seconds).

FIG. 5B is a graph showing the emission scans (302 nm excitation) acquired of reaction mixtures following a 5-minute UV activation period and comprised of: (i) 2,4,6-trichloropyrimidine (0.1%) and lysozyme (0.5 mg/mL) in 1% LDS, (ii) 4,6-dichloro-5-fluoropyrimidine (0.1%) and lysozyme 0.5 mg/mL in 1% LDS, (iii) 2,4,6-trichloropyrimidine (0.1%) and N-acetyl-tryptophan (0.5 mg/mL) in 1% SDS, (iv) TCE (0.1%) and N-acetyl-tryptophan (0.5 mg/mL) in 50% methanol, (v) TCE (0.1%) and N-acetyl-tryptophan (0.5 mg/mL) in 1% SDS, (vi) 4,6-dichloro-5-fluoropyrimidine (0.1%)N-acetyl-tryptophan (0.5 mg/mL) in 50% methanol, and (vii) TCE (0.1%) lysozyme (0.5 mg/mL) in 1% LDS. FIG. 5C is a bar graph showing single wavelength emission data (i.e., relative signal intensities of triplicate readings) of reaction mixtures following a 5-minute UV activation period and comprised of 2,4,6-trichloropyrimidine (0.1%) and lysozyme (0.5 mg/mL) in 1% LDS; 4,6-dichloro-5-fluoropyrimidine (0.1%) and lysozyme (0.5 mg/mL) in 1% LDS; TCE (0.1%) and N-acetyl-tryptophan (0.5 mg/mL) in 50% methanol); 2,4,6-trichloropyrimidine (0.1%) and N-acetyl-tryptophan (0.5 mg/mL) in 1% SDS); 4,6-dichloro-5-fluoropyrimidine (0.1%) and NAT (0.5 mg/mL) in 1% LDS; TCE (0.1%) and NAT (0.5 mg/mL) in 1% LDS. Accordingly, 2,4,6-trichloropyrimidine and 4,6-dichloro-5-fluoropyrimidine exhibited greater signal intensities than TCE.

After acquiring emission scans, the samples in the 96-well plate were electrophoresed and imaged first with no UV activation and then after a UV activation period of 45 seconds. FIG. 5D is a digital image of an electrophoresed Bis-Tris gel with no additional UV activation period (samples had been previously activated for 5 minutes in a 96-well plate prior to loading the samples in the gel), imaged using an auto (optimal) exposure time of 9.526 seconds, and comprising: (i) lysozyme (1.0 μg) in 1% LDS, unlabeled control (Lane 1); (ii) lysozyme (1.0 μg) in 1% LDS, TCE-derivatized (lane 2); (iii) lysozyme (1.0 μg) in 1% LDS, β-butyrolactone (Lane 3); (iv) lysozyme (1.0 μg) in 1% LDS, 2,4,6-trichloropyrimidine-derivatized (Lane 4); (v) lysozyme (1.0 μg) in 1% LDS, 4,6-dichloro-5-fluoropyrimidine-derivatized (Lane 5); (vi) 10 μL Mark12 Unstained Standard (includes reduced/alkylated lysozyme) (Lane 6); (vii) lysozyme (1.0 μg) in 50% MeOH, unlabeled control (Lane 7); (viii) lysozyme (1.0 μg) in 50% MeOH, TCE-derivatized (Lane 8); (ix) lysozyme (1.0 μg) in 50% MeOH, β-butyrolactone (Lane 9); (x) lysozyme (1.0 μg) in 50% MeOH, 2,4,6-trichloropyrimidine-derivatized (Lane 10); (xi) lysozyme (1.0 μg) in 50% MeOH, 4,6-dichloro-5-fluoropyrimidine-derivatized (Lane 11); and (xii) 10 μL Mark12 Unstained Standard (includes reduced/alkylated lysozyme) (Lane 12). FIG. 5E is a digital image of an electrophoresed Bis-Tris gel as a duplicate to that shown in FIG. 5D with no additional UV activation period (samples had been previously activated for 5 minutes in a 96-well plate prior to loading the samples in the gel), imaged using an auto (optimal) exposure time of 9.526 seconds, and comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS (lane 3), β-butyrolactone-derivatized; (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme (1.0 μg) in 50% MeOH, β-butyrolactone-derivatized (lane 9); (x) lysozyme (0.1 μg) in 50% MeOH, 2,4,6-trichloropyrimidine-derivatized (lane 10); (xi) lysozyme (0.1 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

FIG. 5F is a digital image of an electrophoresed Bis-Tris gel with a 45-second UV activation period, imaged using an auto (optimal) exposure time of 6.370 seconds, and comprising: (i) lysozyme (0.1 μg) in 1% LDS, unlabeled control (lane 1); (ii) lysozyme (0.1 μg) in 1% LDS, TCE-derivatized (lane 2); (iii) lysozyme (0.1 μg) in 1% LDS, β-butyrolactone-derivatized (lane 3); (iv) lysozyme (0.1 μg) in 1% LDS, 2,4,6-trichloropyrimidine-derivatized (lane 4); (v) lysozyme (0.1 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (0.1 μg) in 50% MeOH, unlabeled control (lane 7); (viii) lysozyme (0.1 μg) in 50% MeOH, TCE-derivatized (lane 8); (ix) lysozyme (0.1 μg) in 50% MeOH, β-butyrolactone-derivatized (lane 9); (x) lysozyme (0.1 μg) in 50% MeOH, 2,4,6-trichloropyrimidine-derivatized (lane 10); (xi) lysozyme (0.1 μg) in 50% MeOH, 4,6-dichloro-5-fluoropyrimidine-derivatized (lane 11); and (xii) 10 μL Mark12 Unstained Standard (includes reduced/alkylated lysozyme) (lane 12). FIG. 5G is a digital image of an electrophoresed Bis-Tris gel as a duplicate to that shown in FIG. 5D with a 45-second UV activation period, imaged using an auto (optimal) exposure time of 6.370 seconds, and comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme in 50% MeOH, β-butyrolactone-derivatized (1.0 μg) (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

Next, one of the duplicate electrophoresed Bis-Tris gels was Coomassie-stained (SimplyBlue Safe Stain), water de-stained, and imaged. FIG. 5H is the image of the Coomassie-stained (SimplyBlue SafeStain) and water de-stained gel comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% MeOH, TCE-derivatized (lane 8); (ix) lysozyme (1.0 μg) in 50% MeOH, β-butyrolactone-derivatized (lane 9); (x) lysozyme in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

Additionally, one of the duplicate electrophoresed Bis-Tris gels was transferred to a PVDF membrane. FIG. 5I is a digital image of the PVDF membrane with no UV activation, imaged using an auto (optimal) exposure time of 2.764 seconds, and comprising: (i) lysozyme (1.0 μg) in 1% LDS, unlabeled control (lane 1); (ii) lysozyme (1.0 μg) in 1% LDS, TCE-derivatized (lane 2); (iii) lysozyme (1.0 μg) in 1% LDS, β-butyrolactone-derivatized (lane 3); (iv) lysozyme (1.0 μg) in 1% LDS, 2,4,6-trichloropyrimidine-derivatized (lane 4); (v) lysozyme (1.0 μg) in 1% LDS, 4,6-dichloro-5-fluoropyrimidine-derivatized (lane 5); (vi) 10 μL Mark12 Unstained Standard (includes reduced/alkylated lysozyme) (lane 6); (vii) lysozyme (1.0 μg) in 50% MeOH, unlabeled control (lane 7); (viii) lysozyme (1.0 μg) in 50% W MeOH, TCE-derivatized (lane 8); (ix) lysozyme (1.0 μg) in 50% MeOH, β-butyrolactone-derivatized (lane 9); (x) lysozyme (1.0 μg) in 50% MeOH, 2,4,6-trichloropyrimidine-derivatized (lane 10); (xi) lysozyme (1.0 μg) in 50% MeOH, 4,6-dichloro-5-fluoropyrimidine-derivatized (lane 11); and (xii) 10 μL Mark12 Unstained Standard (includes reduced/alkylated lysozyme) (lane 12).

Therefore, in addition to the emission scans of the plate shown in FIGS. 5C-5D, the electrophoresed plate samples comprising 2,4,6-trichloropyrimidine and 4,6-dichloro-5-fluoropyrimidine exhibited a greater signal intensity relative to the plate samples comprising TCE.

### Example 6

In this example, the performance of Bis-Tris gels cast with 2-chloroethanol (0.05%); 2,2,2-trichloroethanol (TCE) (0.05%); and 4,6-dichloro-5-fluoropyrimidine (0.05%) were compared. The gels were loaded and electrophoresed with (i) E. coli lysate (lanes 4-7) at four different load amounts (2.5 μg, 5 μg, 10 μg, and 20 μg), (ii) a five-protein blend (i.e., alcohol dehydrogenase, BSA, carbonic anhydrase, β-lactoglobulin, and lysozyme) at three different load amounts (0.1 μg, 0.2 μg, and 0.4 μg each protein) in duplicate (lanes 1-3 and 9-11), and (iii) 10 μL Mark12 Unstained Standard (lanes 8 and 12) and imaged with no UV activation period, imaged after a UV activation period of 45 seconds, imaged after an additional UV activation period of 5 minutes, and then transferred to PVDF membranes and imaged.

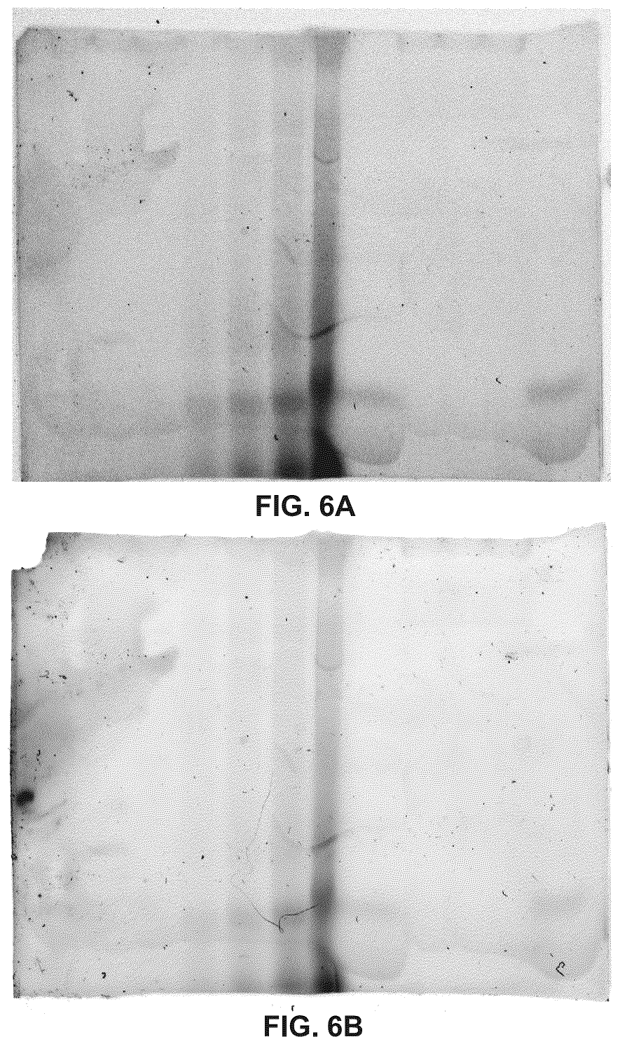

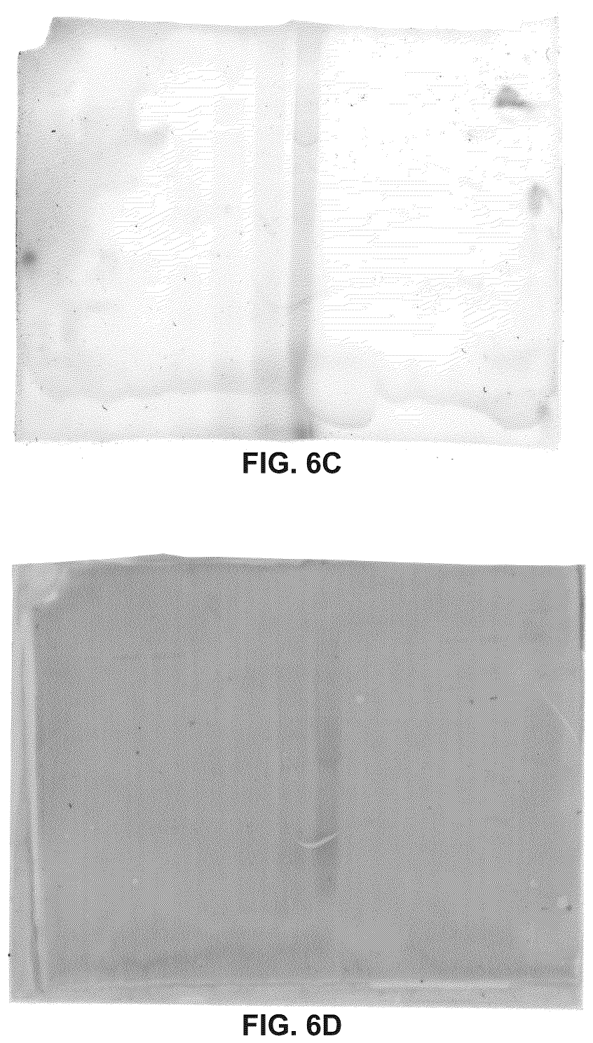

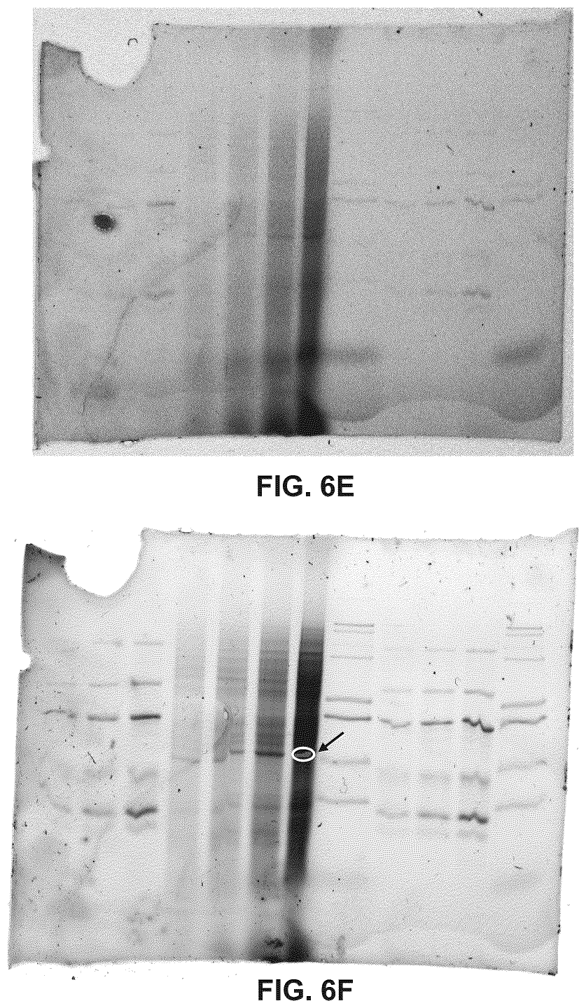

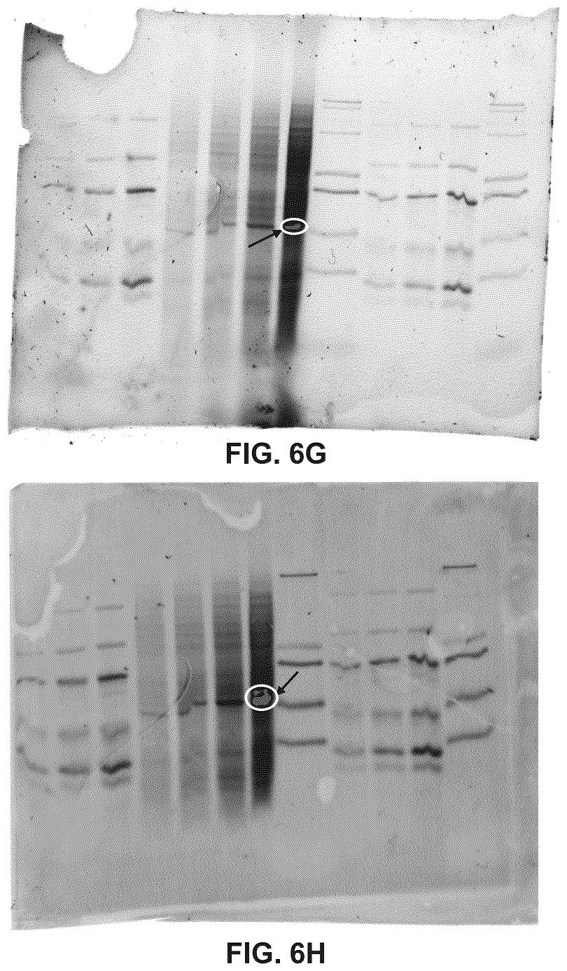

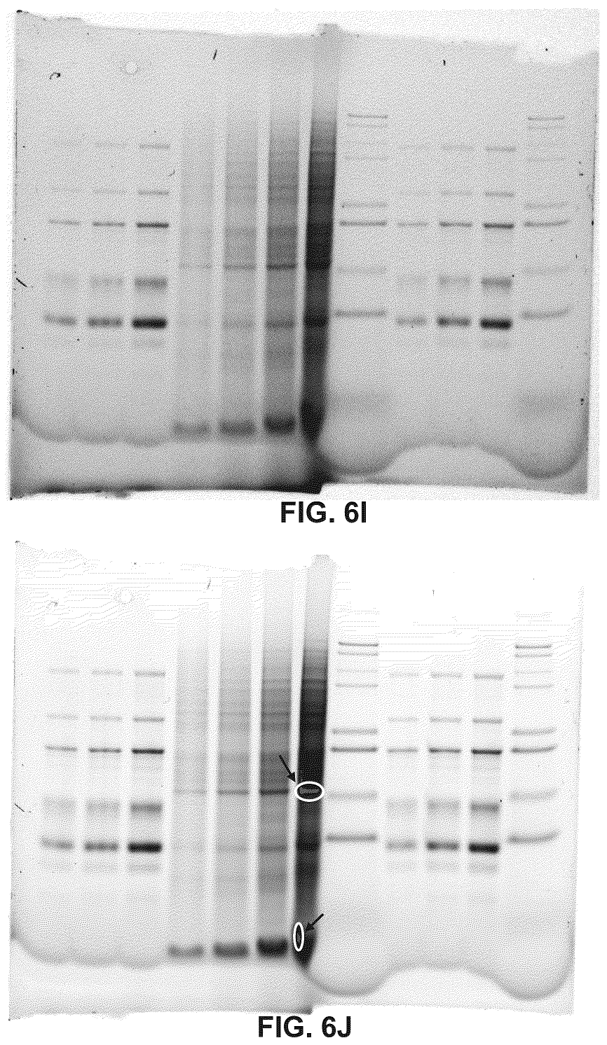

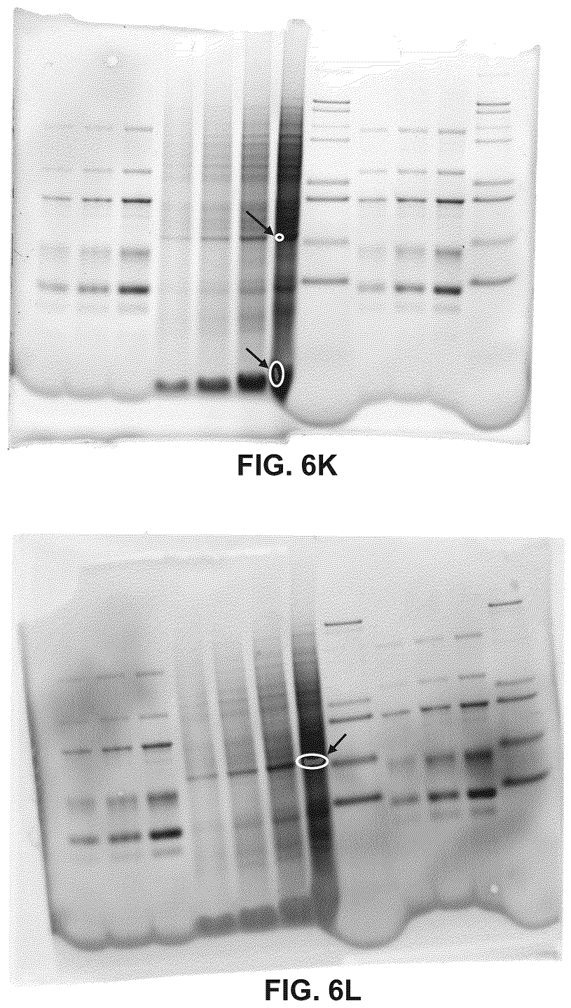

FIG. 6A is a digital image of an electrophoresed Bis-Tris gel (with no UV activation period) comprising 2-chloroethanol (0.05%) and imaged using a manual exposure time of 0.5 seconds. FIG. 6B is a digital image of the same electrophoresed Bis-Tris gel shown in FIG. 6A (but imaged following a 45-second UV activation period) comprising 2-chloroethanol (0.05%) and imaged with the Bis-Tris gel shown in FIG. 6F using an auto (optimal) exposure time of 17.308 seconds. FIG. 6C is a digital image of the same electrophoresed Bis-Tris gel shown in FIG. 6B (but imaged following an additional 5-minute UV activation period) comprising 2-chloroethanol (0.05%) and imaged with the Bis-Tris gel shown in FIG. 6G using auto (optimal) exposure time of 5.385 seconds. FIG. 6D is a digital image of a PVDF membrane (with no further UV activation) after transfer of a Bis-Tris gel (shown in FIG. 6C) and imaged with the membrane shown in FIG. 6H using an auto (optimal) exposure of 2.894 seconds. Accordingly, the gel comprising 2-chloroethanol (0.05%) demonstrated a weak signal which became weaker as the UV activation period increased, and the membrane also demonstrated a weak signal.

FIG. 6E is a digital image of an electrophoresed Bis-Tris gel (with no UV activation period) comprising TCE (0.05%) and imaged using a manual exposure of 0.5 seconds. FIG. 6F is a digital image of the same electrophoresed Bis-Tris gel shown in FIG. 6A (but imaged following a 45-second UV activation period) comprising TCE (0.05%) and imaged with the Bis-Tris gel shown in FIG. 6B an auto (optimal) exposure time of 17.308 seconds. FIG. 6G is a digital image of the same electrophoresed Bis-Tris gel shown in FIG. 6C (but imaged following an additional a 5-minute activation period) comprising TCE (0.05%) and with the Bis-Tris gel shown in FIG. 6D using an auto (optimal) exposure time of 5.385 seconds. FIG. 6H is a digital image of a PVDF membrane (with no further UV activation period) after transfer of a Bis-Tris gel (shown in FIG. 6G) and imaged the membrane shown in FIG. 6D using an auto (optimal) exposure of 2.894 seconds. Accordingly, the gel comprising TCE (0.05%) demonstrated a weak signal (with no UV activation) like the gel comprising 2-chloroethanol (0.05%), wherein the signal became stronger as background decreased with increasing UV activation time.

FIG. 6I is a digital image an electrophoresed Bis-Tris gel (with no UV activation period) comprising 4,6-dichloro-5-fluoropyrimidine (0.05%) and imaged using a manual exposure of 0.5 seconds. FIG. 6J is image of the same electrophoresed Bis-Tris gel shown in in FIG. 6I (but imaged following a 45-second activation period) comprising 4,6-dichloro-5-fluoropyrimidine (0.05%) and imaged using an auto (optimal) exposure time of 5.114 seconds. FIG. 6K is a digital image of the same electrophoresed Bis-Tris gel show in FIG. 6J (but imaged following an additional 5-minute UV activation period) comprising 4,6-dichloro-5-fluoropyrimidine (0.05%) and imaged using an auto (optimal) exposure time of 2.097 seconds. FIG. 6L is a digital image of a PVDF membrane (with no further UV activation) after transfer of a Bis-Tris gel (shown in FIG. 6K) and imaged using an auto exposure of 0.664 seconds. Accordingly, the gels comprising 4,6-dichloro-5-fluoropyrimidine (0.05%) demonstrated a stronger signal intensity with a shorter exposure time and lower background.

Therefore, this example demonstrated that Bis-Tris gels cast with heterocyclic compounds such as, but not limited to, 4,6-dichloro-5-fluoropyrimidine, can be used with lower concentrations relative to 2-chloroethanol and 2,2,2-tricholoroethanol (TCE); and these gels thereby generate a more desirable signal intensities, shorter exposure times, and lower background.

In view of the many possible aspects to which the principles of the present disclosure may be applied, it should be recognized that the illustrated embodiments are only preferred examples of the present disclosure and should not be taken as limiting the scope of the present disclosure. Rather, the scope of the present disclosure is defined by the following claims. We therefore claim as our invention all that comes within the scope and spirit of these claims.

1. A composition for detecting a biomolecule, comprising:
a medium; and
a heterocyclic compound according to Formula I, or an enantiomer, diastereomer, tautomer, salt, solvate, and/or isotopically substituted derivative thereof, 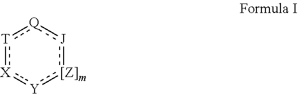
wherein
each of J, Q, T, X, Y, and Z independently is selected from O; P; B; N; S; Se; N(R″) or N⁺[R″], wherein R″ independently is selected from hydrogen, aliphatic, cycloaliphatic, cycloheteroaliphatic, or aryl; or BR^{c} or C(R^{c})ₙ, wherein n is 1 or 2, and
(i) each R^{c} of BR^{c} or C(R^{c})ₙ independently is selected from hydrogen, halo, aliphatic, heteroaliphatic, ether, amino, carbonyl, carboxyl, ketone, aldehyde, isocyanate, cyano, oxime, nitro, nitrile, a salt thereof, or an anionic form thereof, or
(ii) for C(RC)ₙ, two R^{c} groups, together with the carbon atoms to which they are attached, form an aliphatic ring system, a heteroaliphatic ring system, or an aromatic ring system;
provided that at least one of J, Q, T, X, Y, and Z is O, P, N, S, Se, N(R″), N⁺[R″], or BR^{c}; and m is zero is or one; and
wherein the heterocyclic compound is selected to associate with an aromatic amino acid residue of the biomolecule and wherein the heterocyclic compound produces a conjugate upon association with the aromatic amino acid residue of the biomolecule.

2. (canceled)

3. The composition of claim 1, wherein:
(i) m is zero and J and Y are bound by a single or double bond, and the heterocyclic compound has a structure according to Formula II, 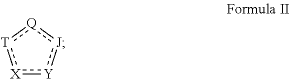
(ii) m is 1, and the heterocyclic compound has a structure according to Formula III.

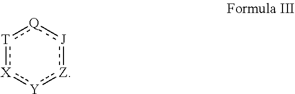

4. The composition of claim 3, wherein the heterocyclic compound has a structure according to Formula II and at least one of J, Q, T, X, or Y is S, Se, O, N, N(R″), N⁺[R′], B, or BR^{c}, and one or more of the remaining J, Q, T, X, or Y groups is CR^{c}, wherein each R^{c} group independently is hydrogen, halo, or cyano.

5. (canceled)

6. The composition of claim 4, wherein the heterocyclic compound is selected from:

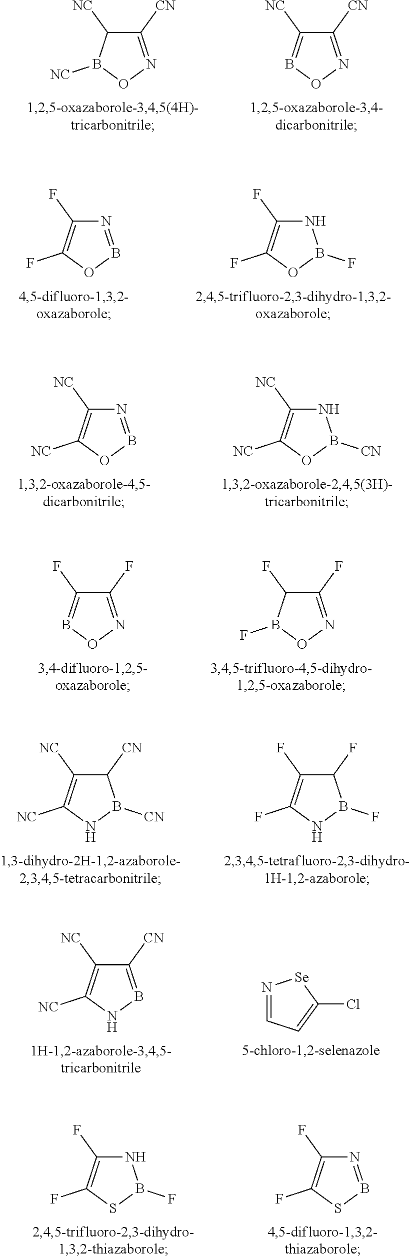

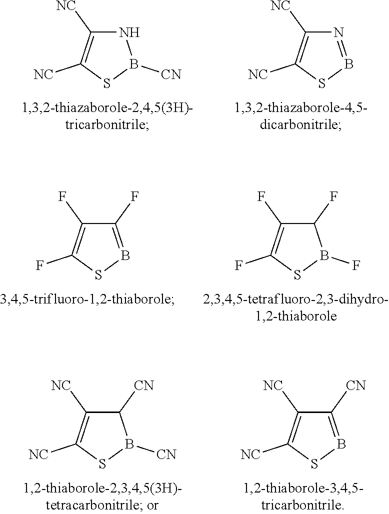 [SMILES: N#CB1NC(C#N)=C(C#N)S1 ; N#Cc1nbsc1C#N ; Fc1bsc(F)c1F ; FB1SC(F)=C(F)C1F ; N#CB1SC(C#N)=C(C#N)C1C#N ; N#Cc1bsc(C#N)c1C#N]

7. The composition of claim 3, wherein the heterocyclic compound has a structure according to Formula II, and where:
(i) two of J, Q, T, X, or Y are N and/or N(R″), wherein R″ is hydrogen; and the remaining J, Q, T, X, or Y groups independently are selected from B; BR^{c}; O; Se; or CR^{c}, wherein each R^{c} independently is hydrogen, cyano, or halo; or
(ii) one or more of J, Q, T, X, or Y is nitrogen: one or more of J, Q, T, X, or Y is N(R″), wherein the R″ group is hydrogen, aliphatic, or cycloaliphatic; and at least two of J, Q, T, X, or Y independently are CR^{c}, wherein the R^{c} groups of the two CR^{c} groups, together with the carbon atoms to which the are bound, form an aryl or heteroaryl ring system.

8. The composition of claim 7, wherein the heterocyclic compound is selected from: 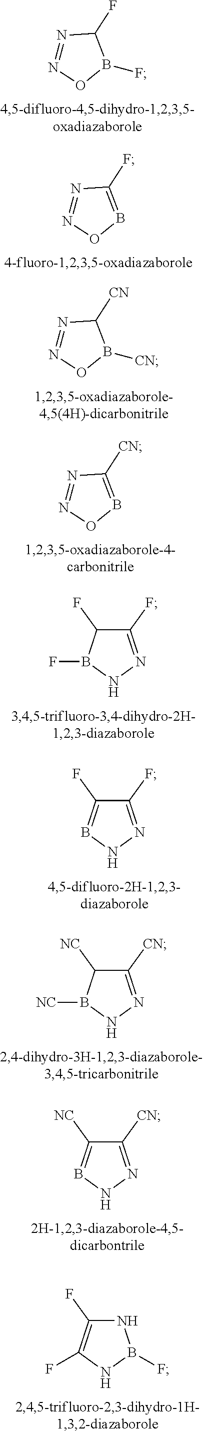 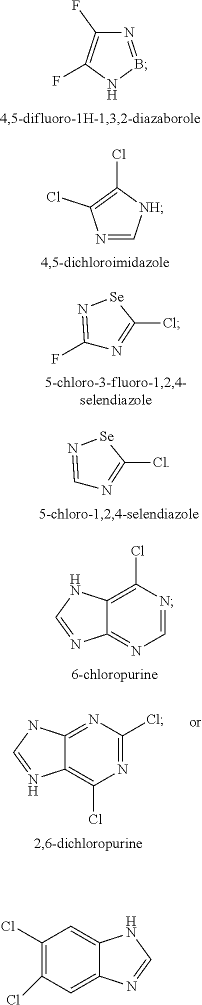
5,6-dichloro-1H-benzo[d]imidazole.

9-15. (canceled)

16. The composition of claim 2, wherein the heterocyclic compound has a structure according to Formula III, and where:
(i) at least one of J, Q, T, X, Y, or Z is N, N(R″), or N⁺[R″] and at least one of J, Q, T, X, Y, or Z is B or BR^{c}, wherein the R^{c} group is halo or cyano, or
(ii) at least one of J, Q, T, X, Y, or Z is N or N⁺[R″] and at least two of J, Q, T, X, Y, or Z are CR^{c}, wherein each R^{c} group independently is selected from halo, heteroaryl, carbonyl, nitro, nitrile, carboxyl, isocyanate, oxime, amino, alkoxy, or aralkyl.

17. (canceled)

18. The composition of claim 16, wherein the heterocyclic compound is selected from:

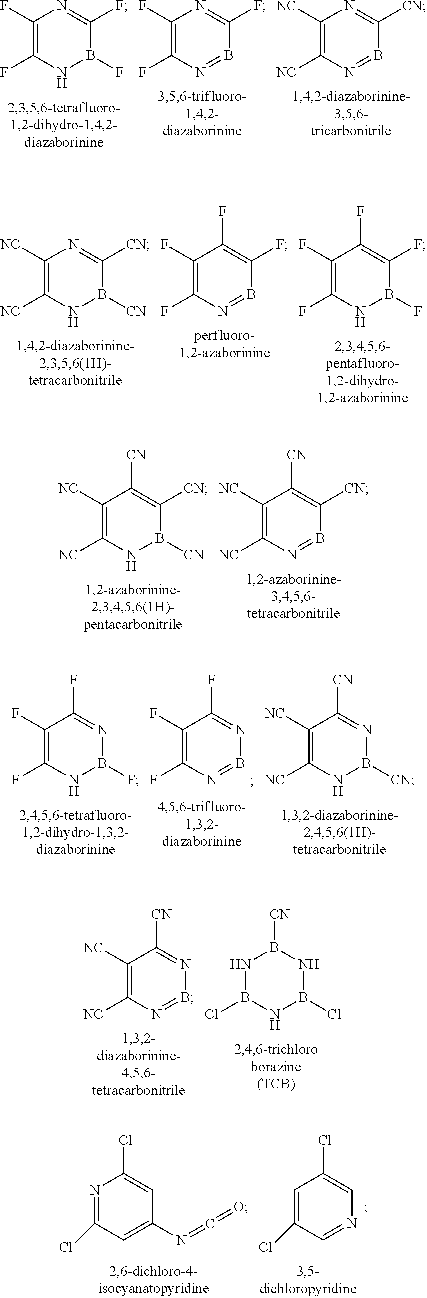

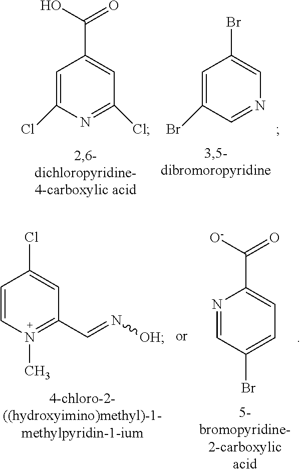

19-22. (canceled)

23. The composition of claim 2, wherein the heterocyclic compound has a structure according to Formula III and at least two of J, Q, T, X, Y, or Z are nitrogen atoms, and wherein the nitrogen atoms are separated by one or two CR^{c} groups.

24-26. (canceled)

27. The composition of claim 23, wherein the nitrogen atoms are separated by one CR^{c} group and the R^{c} group is hydrogen.

28. The composition of claim 27, wherein the heterocyclic compound is selected from:

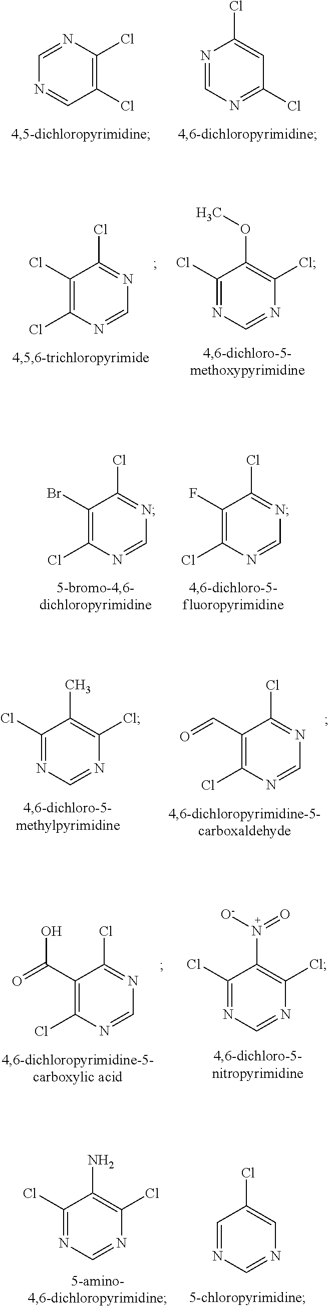

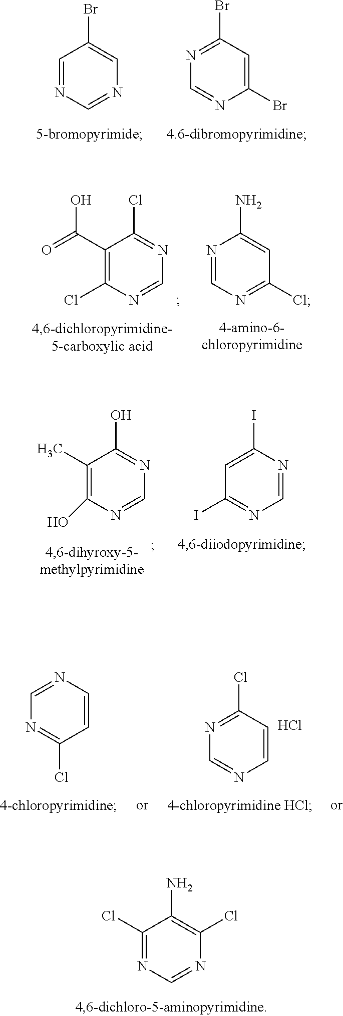

29. The composition of claim 23, wherein the nitrogen atoms are separated by one CR^{c} group and the R^{c} group is other than hydrogen.

30. The composition of claim 29, wherein the heterocyclic compound is selected from:

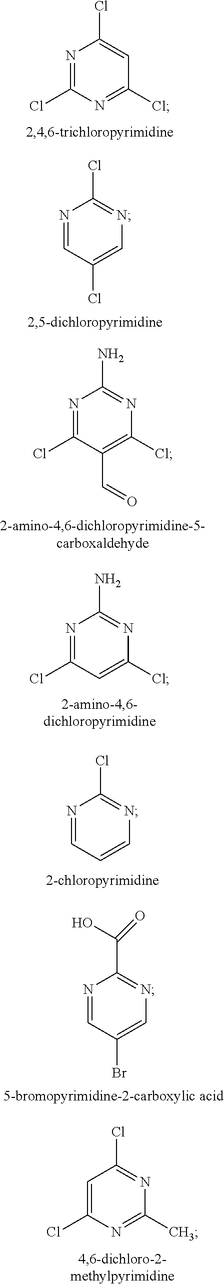

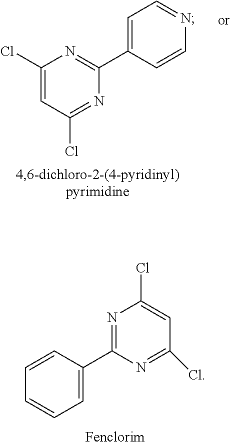 [SMILES: Clc1cc(Cl)nc(-c2ccncc2)n1 ; Clc1cc(Cl)nc(-c2ccccc2)n1]

31. The composition of claim 23, wherein J, Q, T, X, Y, or Z are selected such that two of J, Q, T, X, Y, or Z are nitrogen atoms separated by two CR^{c} groups.

32. The composition of claim 31, wherein the heterocyclic compound is selected from:

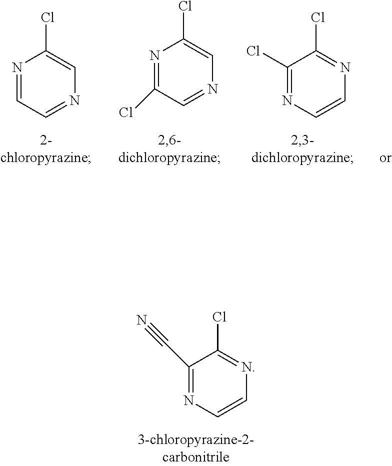

33-39. (canceled)

40. A method for making a composition for detecting one or more proteins in a sample, comprising:
adding the composition of claim 1 to a container;
adding a monomer and a crosslinker to the container;
adding an initiator, promoter, or combination thereof, to the container to promote forming a polymer network derived from the monomer and the crosslinker; and
adding a buffer to the container.

41-45. (canceled)

46. A method for detecting one or more biomolecules in a sample, comprising:
adding the sample to a container comprising (i) the composition of claim 1, (ii) a crosslinked polymer network, and (iii) a buffer;
exposing the container to an energy source; and
detecting an amino acid conjugate formed upon association of the heterocyclic compound with the aromatic amino acid residue of the biomolecule.

47-49. (canceled)

50. A composition comprising:
a buffer;
a polyacrylamide derived from acrylamide and bisacrylamide;
a biomolecule comprising an aromatic amino acid residue; and
an associating means for producing an amino acid conjugate upon associating with the aromatic amino acid residue.

51. A kit for detecting one or more biomolecules, comprising:
a container comprising the composition of claim 1; and instructions for using the composition.

52-53. (canceled)

54. The kit of claim 51, further comprising a buffer.

55. (canceled)


SEQUENCE LISTING (10 sequences)

SEQ ID NO: 1 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 2 - protein, 147 aa, Synthetic construct
  SITE 47: note = X is a modified tryptophan
  SITE 80..81: note = X is a modified tryptophan
  SITE 141: note = X is a modified tryptophan
MRSLLILVLCFLPLAALGKVFGRCELAAAMKRHGLDNYRGYSLGNXVCAAKFESNFNTQATNRNTDGSTDYGILQINSRXXCNDGRTPGSRNLCNIPCSALLSSDITASVNCAKKIVSDGNGMNAWVAWRNRCKGTDVQAXIRGCRL

SEQ ID NO: 3 - protein, 9 aa, Gallus gallus
GTDVQAWIR

SEQ ID NO: 4 - protein, 9 aa, Synthetic construct
  SITE 7: note = X is an acyl 5 modified tryptophan
GTDVQAXIR

SEQ ID NO: 5 - protein, 12 aa, Gallus gallus
GYSLGNWVCAAK

SEQ ID NO: 6 - protein, 12 aa, Synthetic construct
  SITE 7: note = X is an acyl 5 modified tryptophan
GYSLGNXVCAAK

SEQ ID NO: 7 - protein, 12 aa, Synthetic construct
  SITE 7: note = X is an acyl 3 modified tryptophan
GYSLGNXVCAAK

SEQ ID NO: 8 - protein, 12 aa, Synthetic construct
  SITE 7: note = X is an acyl acid modified tryptophan
GYSLGNXVCAAK

SEQ ID NO: 9 - protein, 23 aa, Gallus gallus
NTDGSTDYGILQINSRWWCNDGR

SEQ ID NO: 10 - protein, 23 aa, Synthetic construct
  SITE 17..18: note = X is an acyl acid modified tryptophan
NTDGSTDYGILQINSRXXCNDGR